(19) United States
(12) Patent Application Publication
(10) Pub. No.: US 2007/0009925 A1
(43) Pub. Date: Jan. 11, 2007

(54) GENOMIC DNA SEQUENCING METHODS AND KITS

(75) Inventors: Rixun Fang, Palo Alto, CA (US); Manohar Furtado, San Ramon, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/381,711

(22) Filed: May 4, 2006

Related U.S. Application Data
(60) Provisional application No. 60/678,120, filed on May 5, 2005.

(51) Int. Cl. C12Q 1/68; C12P 19/34

(52) U.S. Cl. 435/6; 435/91.2

(57) ABSTRACT

The disclosed teachings provide methods and kits for determining the sequence of a gDNA target region comprising multiple amplification steps and sequencing at least part of the amplification product of one or more amplification reactions.

### CROSS-REFERENCE TO RELATED APPLICATIONS

This application claims a priority benefit under 35 U.S.C. §119(e) from U.S. Patent Application No. 60/678,120, filed May 5, 2005, which is incorporated herein by reference.

### FIELD

The present teachings generally relate to the fields of biotechnology and molecular biology. More specifically, the disclosed teachings provide methods and kits for sequencing genomic DNA (gDNA) target regions comprising multiple amplification steps, typically starting with only a small amount of gDNA.

### INTRODUCTION

Many molecular biology-based techniques depend on the availability of sufficient quantities of target nucleic acid in the sample being evaluated. For example, many conventional gene analytical methods, for example but not limited to certain sequencing and resequencing methods, require fairly large amounts of starting material, for example in the range of 1-10 microgram (μg) or more of gDNA or RNA. In some circumstances, however, a particular sample may contain only minute amounts of the nucleic acid of interest. For example but not limited to, the small numbers of cells often present in clinical samples obtained by laser capture microscopy; a subpopulation of cells obtained by flow cytometry from a small population containing multiple cell types; clinical swabs, aspirates, or washes; biopsy material such as needle or punch biopsies; or a sample undergoing sterility testing. Additionally, in some instances it may be desired to evaluate the nucleic acid from a single cell. The minute amount of nucleic acid that may be present in such samples presents a challenge, particularly when it is necessary or at least desirable to perform a number of tests, for example, evaluating the sequence of a large number of mutations or single nucleotide polymorphisms (SNPs) in an individual's gDNA. For example, the cystic fibrosis or CFTR gene (approx. 5 kb long), contains approximately 1,300 rare mutations and polymorphisms and it may be desirable to determine the nucleotide sequence at many if not all of the potential mutation and/or SNP sites in a particular individual's gDNA.

The availability of various genomic sequences, including the sequence of the human genome, has assisted in the identification of genetic variation between individuals. Such information has been useful for locating SNP sites, deletion mutations, insertions, and/or translocation sites. In some circumstances, diseases or genetic predisposition to disease have been correlated to such mutation information (see, e.g., Cox et al., Breast Cancer Res. 7:R171-75, 2005; Soranzo et al., Genome Res. 14:1333-44, 2004). Study of the genetic variation between individuals or between clinical isolates has also proved useful for, among other things, evaluating human and microbial evolution (see, e.g., Akey et al., PLOS Biol. 2(10):e286; and Wong et al., Genome Res. 14:398-405, 2004). Such studies depend on the availability of samples comprising adequate amounts of nucleic acid to perform evaluations. Currently, the ability to reliably evaluate samples comprising only small amounts of gDNA is limited and often requires cloning the gDNA to generate sufficient quantities of starting material for sequencing (see, e.g., Venter et al., Science 291:1304-51, 2001; Davison, DNA Seq. 1(6):389-94, 1991; and Claverie, Genomics 23(3):575-81, 1994).

### SUMMARY

The present teachings are directed to methods and kits for sequencing gDNA target regions comprising two or more amplification steps, typically from samples comprising a small amount of gDNA or when only a small amount of gDNA is available for sequencing, for example but not limited to samples comprising 3 nanograms (ng) of gDNA or less, without first cloning the gDNA. According to certain disclosed methods, a first amplification composition is formed comprising a small amount of gDNA, a first extending enzyme, a target-specific primer set for each gDNA target region to be sequenced, and nucleoside triphosphates (NTPs); and under suitable conditions, first amplification products are generated. At least some of the first amplification products are added to a second amplification composition comprising a second extending enzyme and NTPs; and under suitable conditions, a second amplification product is generated. In some embodiments, a third amplification reaction is performed in a third amplification composition comprising at least some of the second amplification products, a third extending enzyme, and NTPs; and under suitable conditions, a third amplification product is generated; and so forth. The step of generating an amplification product in an amplification composition comprising at least some of the amplification product from the previous amplifying step, a suitable extending enzyme, and NTPs can be repeated any number of times, as appropriate. In some embodiments, the amplification product of one amplification reaction is purified before performing the next amplification reaction. The nucleotide sequence of at least part of an amplification product is obtained in a sequencing step, which can but need not include an amplification reaction, and the corresponding sequence of the gDNA target region is determined. In some embodiments, the NTPs in an amplification composition comprise ribonucleoside triphosphates (rNTPs) and/or deoxyribonucleoside triphosphates (dNTPs), including a nucleotide terminator. In some embodiments, at least some of the NTPs in an amplification composition comprise a reporter group. In some embodiments, the nucleotide terminator is a dideoxyribonucleoside triphosphate (ddNTP), including at least one of a ddATP, a ddCTP, a ddGTP, a ddITP, and a ddTTP.

According to certain disclosed methods, an amplifying reaction comprises a DNA-dependent RNA polymerase, an RNA-dependent DNA polymerase, a DNA-dependent DNA polymerase, or combinations thereof. Some disclosed methods comprise a multiplicity of different extending enzymes and a multiplicity of different amplifying steps, for example but not limited to a method comprising an amplifying step comprising a DNA-dependent DNA polymerase, an amplifying step comprising an DNA-dependent RNA polymerase, and an amplifying step comprising a RNA-dependent DNA polymerase and a DNA-dependent DNA polymerase.

Certain embodiments of the disclosed methods include at least one multiplex step, wherein a multiplicity of different gDNA target regions or different amplification products are amplified using a target-specific primer set for each gDNA target region or an amplification product primer set that is specific for each amplification product. Some embodiments of the disclosed methods comprise at least one multiplex reaction and at least one single-plex reaction. In some embodiments, a single-plex reaction comprises a series of massively parallel single-plex reactions. In some embodiments, for each gDNA target region to be sequenced, the first amplification reaction composition comprises one target-specific primer set and a first extending enzyme and the second amplification composition comprises at least some of the first amplification product, one second amplification product primer set, and a second extending enzyme.

Some disclosed methods comprise a limited cycle multiplex first amplification reaction followed by a second amplification reaction. In some embodiments, the first amplification reaction comprises a limited cycle polymerase chain reaction (PCR), for example but not limited to a 5-15 cycle reaction; and a second amplification reaction comprising a longer duration PCR, for example but not limited to a 25-50 cycle reaction, a 25 cycle reaction, a 35 cycle reaction, or a 40 cycle reaction. In some embodiments, the first amplification composition comprises a multiplicity of different target-specific primer sets for amplifying a multiplicity of different gDNA target regions. In some embodiments, the concentration of the target-specific primer sets are very low and may become exhausted before the amplification reaction reaches the plateau phase. In some embodiments, a second amplification reaction is performed in single-plex, including a multiplicity of parallel single-plex reactions, for example but not limited to, wherein each second amplification composition comprises one second amplification product primer set. In some embodiments, there are at least as many different second amplification compositions as there are different gDNA target region primer pairs in the corresponding first amplification composition, wherein each second amplification composition comprises a first amplification product primer set for amplifying one first amplification product species. In some embodiments, at least part of the second amplification product is sequenced to determine the nucleotide sequence of the gDNA target region. In some embodiments, sequencing at least part of the second amplification product comprises a sequencing reaction comprising an extending enzyme.

According to other disclosed methods, a gDNA target region is amplified in a first amplification composition comprising a target-specific primer set and a first amplification product is generated. At least one of the primers of the target-specific primer set comprises a tail sequence comprising a promoter sequence and at least one strand of the first amplification product comprises the promoter sequence or the complement of the promoter sequence. A second amplification composition is formed comprising at least some of the first amplification products comprising the promoter sequence, a DNA-dependent RNA polymerase, and rNTPs; and under suitable conditions, a second amplification product comprising rNTPs is generated. A third amplification composition is formed comprising at least some of the second amplification products, an RNA-dependent DNA polymerase or a DNA-dependent DNA polymerase capable of reverse transcription, and dNTPs. Under suitable conditions, a third amplification product is generated. The third amplification product is contacted with a third amplification product primer pair or at least a third amplification product primer and, under suitable conditions, a fourth amplification product is generated. In some embodiments, the third amplification composition also initially comprises a DNA-dependent DNA polymerase, for example but not limited to a “hot start” DNA polymerase, and a one-step RT-PCR reaction can occur. In other embodiments, the DNA-dependent DNA polymerase is added after the third amplification product is generated and the amplifying comprises a two-step RT-PCR reaction. In some embodiments, at least part of the amplification product is sequenced.

In some embodiments, sequencing at least part of an amplification product comprises forming a sequencing amplification composition comprising at least some of the amplification product, a DNA-dependent DNA polymerase, a sequencing primer or a pair of sequencing primers (e.g., a forward sequencing primer and a reverse sequencing primer), and NTPs; and amplifying the amplification product in the additional amplification composition to generate a sequencing product. The nucleotide sequence of at least part of the sequencing product is obtained and the corresponding sequence of the gDNA target region is determined. In some embodiments, the sequencing composition comprises a reporter group-labeled primer or a reporter group-labeled nucleotide terminator and a reporter group-labeled sequencing product is generated. In some embodiments, the reporter group-labeled amplification product is purified before obtaining at least some of its nucleotide sequence. In some embodiments, sequencing comprises resequencing of human gDNA target regions to evaluate genetic mutations and SNP sites within the gDNA target region.

Kits for performing certain of the instant methods are also disclosed. These and other features of the present teachings are set forth herein.

### DRAWINGS

The skilled artisan will understand that the drawings, described below, are for illustration purposes only. These figures are not intended to limit the scope of the present teachings in any way.

FIG. 1: schematically depicts one illustrative embodiment of the present teachings comprising a first amplifying reaction comprising a limited cycle multiplex PCR, a second amplifying reaction comprising a multiplicity of parallel single-plex PCRs, and a third amplifying reaction comprising a sequencing reaction, and obtaining the nucleotide sequence of at least part of the third amplification product.

FIG. 2: schematically depicts another illustrative embodiment of the current teachings.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example, “a forward primer” means that more than one forward primer can be present, including one or more copies of a particular forward primer species, as well as one or more species of a particular type of forward primer. Also, the use of “comprise”, “comprises”, “comprising”, “contain”, “contains”, “containing”, “include”, “includes”, and “including” are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including but not limited to, patents, patent applications, articles, books, and treatises are expressly incorporated by reference in their entirety for any purpose. In the event that one or more of the incorporated literature and similar materials contradict this application, including the use or meaning of defined terms, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Certain Definitions

The term “affinity tag” as used herein refers to a component of a multi-component complex, wherein the components of the multi-component complex specifically interact with or bind to each other. Some non-limiting examples of multiple-component affinity tag complexes include, ligands and their receptors, for example but not limited to, avidin-biotin, streptavidin-biotin, and derivatives of biotin, streptavidin, or avidin, including, 2-iminobiotin, desthiobiotin, NeutrAvidin (Molecular Probes, Eugene, OR), CaptAvidin (Molecular Probes), and the like; binding proteins/peptides and their binding partners; epitope tags, for example but not limited to c-MYC, HA, VSV-G, and FLAG Tag™, and their corresponding anti-epitope antibodies; haptens, for example but not limited to dinitrophenol (“DNP”) and digoxigenin (“DIG”), and their corresponding antibodies; aptamers and their binding partners; fluorescent reporter groups and corresponding anti-fluorescent reporter group antibodies; and the like.

The term “amplification product” refers to the polynucleotide strand generated by an amplification reaction or a duplex comprising a nucleotide sequence generated by an amplification reaction. An amplification product can be double-stranded, for example but not limited to, a reverse transcription product comprising the RNA template duplexed with the DNA complement of the RNA template. An amplification product can also be a single-stranded polynucleotide for example but not limited to a single-stranded polynucleotide generated by an asymmetric PCR reaction. It is to be understood that the individual strands of a double-stranded amplification product or an individual polynucleotide strand derived from a double-stranded amplification product are also within the intended meaning of the term amplification product, including either or both of the two complementary strands released by denaturing a double-stranded amplification product or the single-stranded DNA obtained by degrading the RNA template component of a cRNA:cDNA duplex generated by reverse transcription. According to the present teachings, an amplification product or at least part of an amplification product is used as a template for a subsequent amplifying step, is used to determine the sequence of the gDNA target region, or both.

The terms “annealing” and “hybridizing”, including variations of the root words hybridize and anneal, are used interchangeably and mean the nucleotide base-pairing interaction of one nucleic acid sequence with another nucleic acid sequence that results in the formation of a duplex, triplex, or other higher-ordered structure. The primary interaction is typically nucleotide base specific, e.g., A:T, A:U, and G:C, by Watson-Crick and Hoogsteen-type hydrogen bonding. Base-stacking and hydrophobic interactions may also contribute to duplex stability.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to a specific relationship between the elements to which the term relates. For example but without limitation, a forward target-specific primer of a particular target-specific primer set corresponds to the reverse target-specific primer of the same target-specific primer set, and vice versa. A primer is designed to selectively hybridize with the primer-binding site of a corresponding amplification product, and vice versa. The target-binding portion of a reverse target-specific primer is designed to selectively hybridize with a complementary or substantially complementary sequence of the corresponding second target flanking region. A particular affinity tag binds to the corresponding affinity tag, for example but not limited to, biotin binding to streptavidin. A particular hybridization tag anneals with its corresponding hybridization tag complement; and so forth.

The terms “denaturing” or “denaturation” as used herein refer to any process in which a double-stranded polynucleotide, for example but not limited to gDNA or certain amplification products, is converted to two single-stranded polynucleotides. Denaturing a double-stranded polynucleotide includes a variety of thermal and chemical techniques for dissociating the two single-stranded components of a duplex. Those in the art will appreciate the denaturing technique employed is generally not limiting unless it inhibits a subsequent annealing or amplifying reaction.

The term “enzymatically active mutants or variants thereof” when used in reference to an enzyme, refers to one or more polypeptide that retains at least some of the desired catalytic activity. It is to be understood that when a particular enzyme or group of enzymes is referred to herein, the enzymatically active mutants of variants of that enzyme or group of enzymes are expressly included.

The term “hybridization tag” as used herein refers to an oligonucleotide sequence that can be used for: separating the element (e.g., amplification products) of which it is a component or to which it is bound, including, bulk separation; tethering or attaching the element to which it is bound to a capture surface, which may or may not include separating; annealing a corresponding hybridization tag complement; or combinations thereof. A “hybridization tag complement” typically refers to an oligonucleotide that comprises a nucleotide sequence that is complementary to and selectively hybridizes with at least part of the corresponding hybridization tag.

The term “mobility modifier” refers any moiety that affects a particular mobility of a polynucleotide in a mobility-dependent analysis technique. The term “mobility-dependent analysis technique” refers to any analysis based on different rates of migration between different analytes. Non-limiting examples of mobility-dependent analysis techniques include electrophoresis, mass spectrometry, chromatography, sedimentation, gradient centrifugation, field-flow fractionation, and multi-stage extraction techniques.

The term “nucleotide terminator” or “terminator” refers to an enzymatically-incorporable nucleotide, which does not support incorporation of subsequent nucleotides in an amplifying reaction and is therefore not an extendable nucleotide.

As used herein, the terms “polynucleotide”, “oligonucleotide”, and “nucleic acid” are used interchangeably and refer to single-stranded and double-stranded polymers of nucleotide monomers, including 2“-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. A polynucleotide may comprise one or more lesions. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymine or an analog thereof, unless otherwise noted.

The term “primer” refers to a polynucleotide that selectively hybridizes to a corresponding target flanking region of a gDNA sequence or a corresponding primer-binding site of an amplification product and allows the synthesis of a sequence complementary to the corresponding polynucleotide template from its 3′ end.

A “universal primer” is capable of selectively hybridizing to the corresponding primer-binding site of more than one species of amplification product. A “universal primer set” comprises a forward universal primer and a reverse universal primer that hybridize with a plurality of species of amplification products. In certain embodiments, a universal primer or a universal primer set selectively hybridizes with all or most of the amplification products in a reaction

As used herein, the term “primer-binding site” refers to a region of a polynucleotide sequence such as a tailed primer or an amplification product that can serve directly, or by virtue of its complement, as the template upon which a primer can anneal for any of a variety of primer extension reactions known in the art (for example, PCR). When a tailed primer comprises a primer-binding site, typically it is located upstream from the sequence-specific binding portion of the primer, for example but not limited to the target-binding portion of a forward target-specific primer or the primer-binding portion of a reverse amplification product-specific primer.

In some embodiments, a primer and/or an amplification product comprises a “promoter sequence”, including a nucleotide segment that, when annealed with its complement forms a double-stranded DNA promoter that is suitable for interacting with a DNA-dependent RNA polymerase, including T3 RNA polymerase, T7 RNA polymerase, or SP6 RNA polymerase. When a tailed primer comprises a promoter sequence, it is typically located upstream from the sequence-specific binding portion of the primer. In some embodiments, a promoter sequence or its complement serves as a primer-binding site, for example but not limited to, a primer-binding site for a sequencing primer.

The term “reporter group” is used in a broad sense herein and refers to any identifiable tag, label, or moiety. The skilled artisan will appreciate that many different species of reporter groups can be used in the present teachings, either individually or in combination with one or more different reporter group.

The term “resequencing” refers to the acts of (a) obtaining the sequence of a gDNA target region for a particular individual and (b) comparing the obtained sequence with a previously known sequence, for example but not limited to a consensus sequence for the gDNA target region or a polymorphic sequence in that gDNA target region. By comparing the obtained sequence with the known gDNA sequence, one can potentially determine the presence or absence of a mutation in that individual at a particular gDNA target region or identify the nucleotide present at a SNP site.

The term “selectively hybridize” and variations thereof means that, under suitable conditions, a given sequence anneals with a second sequence comprising a complementary or a substantially complementary string of nucleotides, but does not anneal to undesired sequences. In this application, a statement that one sequence hybridizes or anneals with another sequence encompasses situations where the entirety of both of the sequences hybridize to one another, and situations where only a portion of one or both of the sequences hybridizes to the entire other sequence or to a portion of the other sequence. For the purposes of this definition, the term “sequence” includes nucleic acid sequences, polynucleotides, oligonucleotides, primers, target-specific portions, primer-binding sites, hybridization tags, and hybridization tag complements.

The term “small amount” when used in reference to the quantity of gDNA in a sample or starting material refers to a minute or limiting quantity of gDNA in that sample or starting material, typically 3 ng of gDNA or less, 2 ng of gDNA or less, 1 ng of gDNA or less, 750 picograms (pg) of gDNA or less, 500 pg of gDNA or less, or 250 pg of gDNA or less, and including all integer quantities of gDNA included therein. Certain samples may contain only a small amount of gDNA, including a clinical specimen such as a micro-biopsy, a tissue or organ wash, or an airway biopsy, or the remainder of an archived sample that may not be re-obtainable, such as certain archeological specimens or certain forensics specimens, including certain crime scene samples.

The term “target region” refers to the gDNA segment that is being amplified and sequenced to determine the identity of a polymorphic nucleotide at a SNP site(s) within the target region, the presence or absence of a mutation within the target region, and so forth. The target region is generally located between two flanking sequences, a first target flanking region and a second target flanking region, located on either side of the target region.

Certain Exemplary Components

According to certain disclosed methods, an amplification composition comprises at least one of an extending enzyme, an ATP sulfurylase, a luciferase, and an apyrase.

The term “extending enzyme” refers to a polypeptide that, under suitable reaction conditions, catalyzes the synthesis of a complementary nucleotide strand in a template-dependent manner. In some embodiments, an extending enzyme catalyzes the 5′-3′extension of a hybridized primer. In some embodiments, an extending enzyme binds to a double-stranded DNA promoter, separates the two strands, and uses the 3′-5′ strand as a template to synthesize a complementary 5′-3′ strand comprising ribonucleotides. Extending enzymes are typically: (1) DNA polymerases, including (a) RNA-dependent DNA polymerases, including reverse transcriptases, and (b) DNA-dependent DNA polymerases; and (2) RNA polymerases, including (a) DNA-dependent RNA polymerases and (b) RNA-dependent RNA polymerases. In certain embodiments, an extending enzyme is a reverse transcriptase, for example but not limited to, retroviral reverse transcriptases such as Avian Myeloblastosis Virus (AMV) reverse transcriptase and Moloney Murine Leukemia Virus (MMLV) reverse transcriptase. In certain embodiments, an extending enzyme is a DNA-dependent DNA polymerase, including Taq DNA polymerase and the Klenow fragment of DNA polymerase I. Certain DNA-dependent DNA polymerases possess reverse transcriptase activity under some conditions, for example but not limited to, the DNA polymerase of Thermus thermophilus (Tth DNA polymerase, E.C. 2.7.7.7) which demonstrates reverse transcription in the presence of Mn²⁺, but not Mg²⁺ (see also, GeneAmp® AccuRT RNA PCR Kit and Hot Start RNA PCR Kit comprising a recombinant polymerase derived from Thermus species Z05, both from Applied Biosystems). Likewise, certain reverse transcriptases possess DNA-dependent DNA polymerase activity under certain reaction conditions, including AMV reverse transcriptase and MMLV reverse transcriptase. In some embodiments, an amplification reaction comprises transcription, including in vitro transcription, and an extending enzyme comprises a DNA-dependent RNA polymerase, for example but not limited to bacteriophage T3, SP6, and T7 RNA polymerases. Descriptions of extending enzymes can be found in, among other places, Lehninger Principles of Biochemistry, 3d ed., Nelson and Cox, Worth Publishing, New York, N.Y., 2000 (“Lehninger”), particularly Chapters 26 and 29; Twyman, Advanced Molecular Biology: A Concise Reference, Bios Scientific Publishers, New York, N.Y., 1999; Ausubel et al., Current Protocols in Molecular Biology, John Wiley & Sons, Inc., including supplements through April 2005 (“Ausubel et al.“); and Enzymatic Resource Guide: Polymerases, Promega, Madison, Wis., 1998. Expressly within the intended scope of the term extending enzyme are enzymatically active mutants or variants thereof, and incluidng enzymes modified to confer different temperature-sensitive properties (see, e.g., U.S. Pat. Nos. 5,773,258; 5,677,152; and 6,183,998; and DNA Amplification: Current Techniques and Applications, Demidov and Broude, eds., Horizon Bioscience, 2004, particularly in Chapter 1.1).

An “apyrase” is an polypeptide that, under suitable conditions, converts NTPs into nucleoside monophosphates and phosphate, i.e., dNTP to dNMP+2 Pi (see, e.g., Agah et al., Nucl. Acids Res. 32:e166, 2004). Expressly within the intended scope of the term apyrase are enzymatically active mutants or variants thereof.

The term “ATP sulfurylase”, also known as sulfate adenylyltransferase, refers to a polypeptide that, under suitable conditions, catalyzes the reaction: ATP+sulfate=pyrophosphate+adenylyl sulfate (see, e.g., Nyren and Lundin, Analyt. Biochem. 151:504-09, 1985; and Agah et al., Nucl. Acids Res. 32:e166, 2004). Expressly within the intended scope of the term ATP sulfurylase are enzymatically active mutants or variants thereof.

A “luciferase” is a polypeptide that, under suitable conditions, catalyzes the conversion of ATP, luciferin, and oxygen (O₂) to AMP, CO₂, oxyluciferin, PPi, and light (see, e.g., Agah et al., Nucl. Acids Res. 32:e166, 2004; and Nyren, Analyt. Biochem. 167:235-38, 1987). In some embodiments, a polypeptide, such as a fusion protein, comprises ATP sulfurylase and luciferase activity (see, e.g., U.S. Patent Application Publication US 2003/0113747). Expressly within the intended scope of the term luciferase are enzymatically active mutants or variants thereof.

An enzymatically active mutant or variant of a given enzyme is a polypeptide that differs from the enzyme in some way, but retains at least some of the desired catalytic activity. For example, some enzymatically active mutants or variants of Thermus aquaticus (Taq) DNA-dependent DNA polymerase include AmpliTaq® DNA polymerase, AmpliTaq Gold® DNA polymerase, the Stoffel fragment of AmpliTaq® DNA polymerase, and AmpliTaq® DNA polymerase CS (Applied Biosystems).

Also within the scope of this term are: enzymatically active fragments, including, cleavage products, for example but not limited to Klenow fragment, Stoffel fragment, or recombinantly expressed fragments and/or polypeptides that are smaller in size than the corresponding enzyme; mutant forms of the corresponding enzyme, including but not limited to, naturally-occurring mutants, such as those that vary from the “wild-type” or consensus amino acid sequence, mutants that are generated using physical and/or chemical mutagens, and genetically engineered mutants, for example but not limited to random and site-directed mutagenesis techniques; amino acid insertions and deletions, and changes due to nucleic acid nonsense mutations, missense mutations, and frameshift mutations; reversibly modified enzymes, for example but not limited to those described in U.S. Pat. No. 5,773,258; biologically active polypeptides obtained from gene shuffling techniques (see, e.g., U.S. Pat. Nos. 6,319,714 and 6,159,688), splice variants, both naturally occurring and genetically engineered, provided that they are derived, at least in part, from one or more corresponding enzymes; chimeric enzymes, including fusion proteins (see, e.g., DNA Amplification, Demidov and Broude, eds., Horizon Biosciences, 2004; and U.S. Patent Application Publication Nos. US 2003/0113747 A1 and US 2003/0119012 A1); polypeptides corresponding at least in part to one or more such enzymes that comprise modifications to one or more amino acids of the native sequence, including, adding, removing or altering glycosylation, disulfide bonds, hydroxyl side chains, and phosphate side chains, or crosslinking, provided such modified polypeptides retain at least some of the desired catalytic activity; and the like. Expressly within the meaning of the term “enzymatically active mutants or variants thereof” when used in reference to a particular enzyme(s) are enzymatically active mutants of that enzyme, enzymatically active variants of that enzyme, or enzymatically active mutants of that enzyme and enzymatically active variants of that enzyme.

The skilled artisan will readily be able to measure catalytic activity using an appropriate assay known in the art. Thus, an appropriate assay for DNA-dependent DNA polymerase catalytic activity might include, for example, measuring the ability of a variant to incorporate, under appropriate conditions, dNTPs into a polynucleotide strand in a template-dependent manner. Likewise, an appropriate assay for DNA-dependent RNA polymerase activity might include, for example, the ability to bind to a promoter sequence and synthesize a complementary RNA strand in a template-dependent manner. Descriptions of some relevant assays may be found in, among other places, Sambrook and Russell, Molecular Cloning, A Laboratory Manual, Cold Spring Harbor Press, 3d ed., 2001 (“Sambrook and Russell”); Sambrook, Fritsch, and Maniatis, Molecular Cloning, A Laboratory Manual, Cold Spring Harbor Press, 2d ed., 1989 (“Sambrook et al.”); Ausubel et al.; and Ronaghi, Genome Res. 11:3-11, 2001. It is to be understood that enzymatically active mutants or variants thereof are expressly within the scope of the current teachings.

The first amplification compositions of the current teachings comprise gDNA, typically a small amount of gDNA, including at least one target region located between a corresponding first flanking region and a second flanking region. The “first target flanking region” is typically located upstream from, i.e., on the 5′ side of, the target region and the corresponding “second target flanking region” is typically located downstream from, i.e., on the 3′ side of, the target region. For illustration purposes, the orientation of an illustrative target region relative to its two target flanking regions is: 5′-first target flanking region-target region-second target flanking region-3′. It is to be understood that the target flanking regions can, but need not, be contiguous with the target region. Thus, additional nucleotides may be present between a target flanking region and the target region. The target-binding portion of the forward target-specific primer comprises a sequence that is designed to selectively hybridize with the complement of the first target flanking region or a sequence within the first target flanking region. The target-binding portion of the reverse target-specific primer comprises a sequence that is designed to selectively hybridize with the second target flanking region or a sequence within the second target flanking region. In some embodiments, a gDNA segment comprises a plurality of target regions. In some embodiments, a target is contiguous with or adjacent to one or more additional target regions. In some embodiments, a given target region can overlap a first target region on its 5′-end, a second target region on its 3′-end, or both.

Certain amplification compositions of the current teachings comprise a primer set, for example but not limited to a target-specific primer set, a second amplification product primer set, a third amplification product primer set, a fourth amplification product primer set, a fifth amplification product primer set, a sequencing primer set, or combinations thereof. In some embodiments, a primer or a sequencing primer is employed to generate a single-stranded amplification product, for example but not limited to a single-stranded amplification product or a family of sequencing fragments. Primer sets of the current teachings typically comprise a forward primer and a corresponding reverse primer. In some embodiments, a target-specific primer set further comprises a second forward primer, e.g., a forward target-specific primer, a corresponding second forward primer, and a corresponding reverse target-specific primer.

In some embodiments, a primer comprises a tail portion comprising a primer-binding site or its complement, a promoter sequence or its complement, an affinity tag, a hybridization tag, a mobility modifier, or combinations thereof. In some embodiments, a promoter sequence comprises a multiplicity of different promoter sequences, for example but not limited to, a T7 RNA polymerase binding site, an SP6 RNA polymerase binding site, and a T3 polymerase binding site.

In some embodiments, a primer does not comprise a tail portion. In some embodiments, a primer comprises a reporter group, an affinity tag, a primer-binding site or its complement, a promoter sequence or its complement, a hybridization tag, a mobility modifier, or combinations thereof.

In some embodiments, a primer-binding site comprises a universal priming sequence or its complement, allowing at least some amplification products to be generated using a universal primer or a universal primer set. In some embodiments, a sequencing primer comprises a universal priming sequence. Universal primers/priming sequences (sometimes referred to as common or generic primers), including M13 universal primers and T7 universal primers, and their use are well known in the art (see, e.g., McPherson, particularly section 4.2 of Chapter 5). In some embodiments, a universal primer or a pair of universal primers can be employed as sequencing primers for a sequencing reaction; and either or both strands of a double-stranded amplification product can be sequenced. Universal primers are commercially available from numerous vendors including Applied Biosystems, USB Corporation, Invitrogen, and Promega. Those in the art will understand that “custom” universal primers can also be designed and synthesized using methods known in the art.

It will be appreciated by those of skill in the art that when two corresponding primer-binding sites are present on a single polynucleotide (for example but not limited to, a single-stranded amplification product, or a single strand obtained by denaturing or degrading a double-stranded amplification product), the orientation of the two primer-binding sites is generally different. For example, one primer of a primer set is complementary to and can selectively hybridize with one of the two primer-binding sites, while the corresponding primer of the primer set is designed to selectively hybridize with the complement of the other of the two primer-binding sites. Stated another way, in some embodiments one primer-binding site on a single-stranded nucleic acid sequence can be in a sense orientation, and the corresponding primer-binding site on the same nucleic acid can be in an antisense orientation.

As used herein, “forward” and “reverse” are used to indicate relative orientation of corresponding primers of a primer set on a polynucleotide sequence. For illustration purposes but not as a limitation, consider a single-stranded polynucleotide drawn in a horizontal, left to right, orientation with its 5′-end on the left. The “reverse” primer is designed to selectively hybridize with the downstream primer-binding site at or near the 3′- or right end of this illustrative polynucleotide. The corresponding “forward primer is designed to selectively hybridize with the complement of the upstream primer-binding site at or near the 5′- or left end of the polynucleotide. Thus, the reverse primer comprises a sequence that is complementary to or substantially complementary to the second or downstream primer-binding site of the polynucleotide and the forward primers comprises a sequence that is the same as or substantially the same as the first or upstream primer-binding site. It is to be understood that the terms “3-end” and “5′-end”, as used in this paragraph, are illustrative only and do not necessarily refer literally to the respective ends of the polynucleotide. Rather, the only limitation is that the reverse primer of this exemplary primer set selectively hybridizes with a reverse primer-binding site that is downstream or to the right of the forward primer-binding site that comprises the same sequence or substantially the same sequence as at least part of the corresponding forward primer. As will be recognized by those of skill in the art, these terms are not intended to be limiting, but rather to provide illustrative orientation in a given embodiment.

Those in the art appreciate that as an amplification product is amplified by certain amplification techniques, the complement of the primer-binding site is synthesized in the complementary amplicon. Thus, it is to be understood that the complement of a primer-binding site is expressly included within the intended meaning of the term primer-binding site, unless stated otherwise.

Conditions under which primers selectively hybridize to complementary or substantially complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349, 1968. In general, whether such annealing takes place is influenced by, among other things, the length of the complementary portion of the primers and their corresponding target flanking regions or the corresponding primer-binding sites in amplification products, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or minor groove binders in the sequence-specific portion of a primer and/or a corresponding amplification product can also influence hybridization conditions. Thus, the preferred annealing conditions will depend upon the particular application. Such conditions, however, can be routinely determined by persons of ordinary skill in the art, without undue experimentation. Typically, annealing conditions are selected to allow the disclosed primers to selectively hybridize with a complementary or substantially complementary sequence in corresponding target flanking region or corresponding amplification product, but not hybridize to any significant degree to other undesired sequences in the reaction.

The criteria for designing sequence-specific primers are well known to persons of ordinary skill in the art. Descriptions of primer design can be found in, among other places, Diffenbach and Dveksler, PCR Primer, A Laboratory Manual, Cold Spring Harbor Press (1995); Rapley; and Kwok et al., Nucl. Acid Res. 18:999-1005 (1990). Primer design software programs are also commercially available, for example, Primer Premier 5, PREMIER Biosoft, Palo Alto, Calif.; Primer Designer 4, Sci-Ed Software, Durham, N.C.; Primer Detective, ClonTech, Palo Alto, Calif.; Lasergene, DNASTAR, Inc., Madison, Wis.; and iOligo, Caesar Software, Portsmouth, N.H.

The skilled artisan will appreciate that while the primers and primer sets of the present teachings may be described in the singular form, a plurality of primers may be encompassed by the singular term. Thus, for example, in certain embodiments, a target-specific primer set typically comprises a plurality of forward target-specific primers and a plurality of corresponding reverse target-specific primers; and in some embodiments, a plurality of corresponding second forward primers.

In some embodiments, a multiplicity of different primer sets are employed in an amplifying step, for example but not limited to a multiplex amplification reaction, wherein the different primer sets are designed to amplify a multiplicity of different nucleotide sequences, including a multiplicity of different gDNA target regions or a multiplicity of different amplification products. In some embodiments, a primer set comprises an target-specific primer set, including (1) a forward target-specific primer comprising (a) a first target-binding portion that is the same as or substantially the same as a first target flanking sequence, located upstream (5′) of the gDNA target region and (b) a first tail portion located upstream from the first target-binding portion, wherein the tail sequence comprises a first primer-binding site, a first promoter sequence, or a first primer-binding site and a first promoter sequence; and (2) a corresponding reverse target-specific primer comprising (a) a second target-binding portion that is complementary to or substantially complementary to a corresponding second target flanking sequence, located downstream (3′) of the same gDNA target region and (b) a second tail sequence located upstream from the second target-binding sequence, wherein the second tail sequence comprises a second primer-binding site, a second promoter sequence, or a second primer-binding site and a second promoter sequence.

In some embodiments, a target-specific primer set comprises (1) a forward target-specific primer comprising a first target-binding portion that is the same as or substantially the same as a first target flanking sequence, located upstream (5′) of the gDNA target region and (2) a corresponding reverse target-specific primer comprising a second target-binding portion that is complementary to or substantially complementary to a corresponding second target flanking sequence, located downstream (3′) of the same gDNA target region. In some embodiments, the forward target-specific primer further comprises a first tail portion that is located upstream from the first target-binding portion, wherein the first tail portion comprises a primer-binding site, a promoter sequence, or a primer-binding site and a promoter sequence.

In some embodiments, a target-specific primer set comprises three different primers, including a forward target-specific primer, a second forward primer, and a reverse target-specific primer. The forward target-specific primer comprises (a) a first target-binding portion that is the same as or substantially the same as a first target flanking sequence, located 5′ of the gDNA target region and (b) a first tail portion located upstream from the first target-binding portion, wherein the tail sequence comprises a first primer-binding site. The corresponding second forward primer comprises (a) a sequence that is complementary to or substantially complementary to and is designed to selectively hybridize with the first primer-binding site of the first forward primer or to selectively hybridize with the complement of the first primer-binding site of the first forward primer and (b) a promoter sequence. The corresponding reverse target-specific primer comprises (a) a second target-binding sequence that is complementary to or substantially complementary to the corresponding second target flanking sequence, located 3′ of the same gDNA target region and (b) a second tail portion located upstream from the second target-binding sequence, wherein the second tail portion comprises a second primer-binding site. In some embodiments, the second forward primer is a universal primer in that it is a member of a multiplicity of different target-specific primer sets.

In some embodiments, a primer set comprises an amplification product primer set comprising a forward amplification product primer and a reverse amplification product primer, including a first amplification product primer set, a second amplification product primer set, a third amplification product primer set, and so forth, as appropriate. In some embodiments, an amplification primer set comprises a universal primer or a universal primer set and the same primer set is used to selectively amplify at least two different species of amplification product. In some embodiments, an amplification product primer set comprises a forward primer and a reverse primer that are designed to amplify one amplification product species. For example but without limitation, a first amplification product primer set comprising a forward first amplification product primer comprising a sequence that is designed to selectively hybridize with the complement of an upstream primer-binding site of a particular single-stranded first amplification product species and a reverse first amplification product primer that is designed to selectively hybridize with the corresponding downstream primer-binding site of the same single-stranded first amplification product species. In some embodiments, the primers of an amplification product primer set are designed to selectively hybridize with the same primer-binding sequences as a primer set employed in a previous amplification reaction or the complement of those primer-binding sequences. In some embodiments, an amplification product primer set is designed to selectively hybridize with corresponding regions of the amplification product that are internal to the binding sites of the previous primer set, including a nested primer set, or that partially overlap the binding sites of the previous primer set.

The skilled artisan will appreciate that the complement of the disclosed gDNA target regions, primers, target-binding portions, primer-binding sites, promoter sequences, or combinations thereof, may be employed in certain embodiments of the present teachings. For example, without limitation, a particular gDNA may comprise both the gDNA target region and its complement. Thus, in certain embodiments, when a gDNA sample is denatured, both the target region and its complement are present in the sample as single-stranded sequences and either or both of the single-stranded sequences can be sequenced and analyzed.

In some embodiments, a primer comprises a reporter group, an amplification product comprises a reporter group, an amplification composition comprises a reporter group, or combinations thereof. In certain embodiments, a reporter group emits a fluorescent, a chemiluminescent, a bioluminescent, a phosphorescent, or an electrochemiluminescent signal. Some non-limiting examples of reporter groups include fluorophores, radioisotopes, chromogens, enzymes, antigens including but not limited to epitope tags, semiconductor nanocrystals such as quantum dots, heavy metals, dyes, phosphorescence groups, chemiluminescent groups, electrochemical detection moieties, binding proteins, phosphors, rare earth chelates, transition metal chelates, near-infrared dyes, electrochemiluminescence labels, and mass spectrometer-compatible reporter groups, such as mass tags, charge tags, and isotopes (see, e.g., Haff and Smirnov, Nucl. Acids Res. 25:3749-50, 1997; Xu et al., Anal. Chem. 69:3595-3602, 1997; Sauer et al., Nucl. Acids Res. 31:e63, 2003).

The term reporter group also encompasses an element of multi-element reporter systems, including, affinity tags such as biotin:avidin, antibody:antigen, and the like, in which one element interacts with one or more other elements of the system in order to effect the potential for a detectable signal. Some non-limiting examples of multi-element reporter systems include an oligonucleotide comprising a biotin reporter group and a streptavidin-conjugated fluorophore, or vice versa; an oligonucleotide comprising a DNP reporter group and a fluorophore-labeled anti-DNP antibody; and the like. Detailed protocols for attaching reporter groups to nucleic acids can be found in, among other places, Hermanson, Bioconjugate Techniques, Academic Press, San Diego, 1996; Current Protocols in Nucleic Acid Chemistry, Beaucage et al., eds., John Wiley & Sons, New York, N.Y. (2000), including supplements through April 2005; and Haugland, Handbook of Fluorescent Probes and Research Products, 9^{th} ed., Molecular Probes, 2002.

Multi-element interacting reporter groups are also within the intended scope of the term reporter group, such as fluorophore-quencher pairs, including fluorescent quenchers and dark quenchers (also known as non-fluorescent quenchers). A fluorescent quencher can absorb the fluorescent signal emitted from a fluorescent reporter group and after absorbing enough fluorescent energy, the fluorescent quencher can emit fluorescence at a characteristic wavelength, e.g., fluorescent resonance energy transfer (FRET). For example without limitation, the FAM-TAMRA pair can be illuminated at 492 nm, the excitation peak for FAM, and emit fluorescence at 580 nm, the emission peak for TAMRA. In some embodiments, an extending enzyme comprises a fluorescent reporter group, such as a FRET donor and a NTP comprises a fluorescent quencher (see, e.g., U.S. Published Patent Application No. US 2003/0064366 A1). A dark quencher, appropriately paired with a fluorescent reporter group, absorbs the fluorescent energy from the fluorophore, but does not itself fluoresce. Rather, the dark quencher dissipates the absorbed energy, typically as heat. Some non-limiting examples of dark or nonfluorescent quenchers include Dabcyl, Black Hole Quenchers, Iowa Black, QSY-7, AbsoluteQuencher, Eclipse non-fluorescent quencher, metal clusters such as gold nanoparticles, and the like. Certain dual-labeled probes comprising fluorescent reporter group-quencher pairs can emit fluorescence when the members of the pair are physically separated, for example but without limitation, nuclease probes such as TaqMan® probes. Other dual-labeled probes comprising fluorescent reporter group-quencher pairs can emit fluorescence when the members of the pair are spatially separated, for example but not limited to hybridization probes such as molecular beacons or extension probes such as Scorpion primers. Fluorophore-quencher pairs are well known in the art and used extensively for a variety of reporter probes (see, e.g., Yeung et al., BioTechniques 36:266-75, 2004; Dubertret et al., Nat. Biotech. 19:365-70, 2001; and Tyagi et al., Nat. Biotech. 18:1191-96, 2000).

In some embodiments, a primer and/or an amplification product comprise an affinity tag. In some embodiments, an affinity tag comprises a reporter group. In certain embodiments, affinity tags are used for separating, are part of a detecting means, or both.

In some embodiments, a primer and/or an amplification product comprises a hybridization tag, a hybridization tag complement, or both. In certain embodiments, the same hybridization tag is used with a multiplicity of different elements to effect bulk separation and/or capture surface attachment, for example but not limited to certain hybridization-based pullout formats (see, e.g., ABI PRISM® Duplex™ 384 Well F/R Sequence Capture Kit, Applied Biosystems). In various embodiments, hybridization tag complements serve as capture moieties for attaching a hybridization tag:element complex to a capture surface, for example but not limited to a particular address or location on a microarray or bead array; serve as “pull-out” sequences for bulk separation procedures or hybridization-based pullout; or both as capture moieties and as pull-out sequences. In certain embodiments, a hybridization tag complement comprises a reporter group, a mobility modifier, a reporter probe-binding portion (for example but not limited to a sequence that selectively hybridizes with a TaqMan® probe or other nuclease probe, a molecular beacon probe or other hybridization probe, a scorpion primer or other extension primer, and so forth), or combinations thereof. In certain embodiments, a hybridization tag complement is annealed to a corresponding hybridization tag and, subsequently, at least part of that hybridization tag complement is released and detected.

Typically, hybridization tags and their corresponding hybridization tag complements are selected to minimize: internal self-hybridization or cross-hybridization with different hybridization tag species, nucleotide sequences in an amplification composition, including but not limited to gDNA, different species of hybridization tag complements, primers, primer-binding sites or promoter sequences of amplification products, and the like; but should be amenable to facile hybridization between the hybridization tag and its corresponding hybridization tag complement. In some embodiments, however, a primer-binding site or a promoter sequence of an amplification product, or at least part of these sequences, can serve as a hybridization tag for the amplification product (see, e.g., ABI PRISM® DupleX™ 384 Well F/R Sequence Capture Kit, Applied Biosystems). Hybridization tag sequences and hybridization tag complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65, 1998). Descriptions of hybridization tags, hybridization tag complements, and their use can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein); Gerry et al., J. Mol. Biol. 292:251-262, 1999) (referred to as “zip-codes” and “zip-code complements” therein); and Brenner et al., Proc. Natl. Acad. Sci. 97:1665-70, 2000 (referred to as “oligonucleotide tags”, “tags”, and “anti-tags” therein). Those in the art will appreciate that a hybridization tag and its corresponding hybridization tag complement are, by definition, complementary to each other and that the terms hybridization tag and hybridization tag complement are relative and can essentially be used interchangeably in most contexts.

Hybridization tags can be located at or near the end of a primer and/or an amplification product; or they can be located internally. In certain embodiments, a hybridization tag is attached to a primer and/or an amplification product via a linker arm. In certain embodiments, the linker arm is cleavable.

In certain embodiments, hybridization tags are at least 12 bases in length, at least 15 bases in length, 12-60 bases in length, or 15-30 bases in length. In certain embodiments, a hybridization tag is 12, 15, 20, 21, 22, 23, 24, 25, 26, 27, 28, 29, 30, 45, or 60 bases in length. In certain embodiments, at least two hybridization tag:hybridization tag complement duplexes have melting temperatures that fall within a Δ Tₘ range (Tₘₐₓ-Tₘᵢₙ) of no more than 10° C. of each other. In certain embodiments, at least two hybridization tag:hybridization tag complement duplexes have melting temperatures that fall within a Δ Tₘ range of 5° C. or less of each other.

In some embodiments, a primer and/or an amplification product comprise a mobility modifier. In certain embodiments, mobility modifiers comprise nucleotides of different lengths effecting different mobilities. In certain embodiments, mobility modifiers comprise non-nucleotide polymers, for example but not limited to, polyethylene oxide (PEO), polyglycolic acid, polyurethane polymers, polypeptides, and oligosaccharides. In certain embodiments, mobility modifiers may work by adding size to a polynucleotide, or by increasing the “drag” of the molecule during migration through a medium without substantially adding to the size. Certain mobility modifiers, including PEO's, have been described in, among other places, U.S. Pat. Nos. 5,470,705; 5,580,732; 5,624,800; and 5,989,871 and U.S. Patent Application Publication No. US 2003/0190646 A1.

In some embodiments, an amplification composition comprises a nucleotide terminator, also referred to as a terminator, particularly when the amplifying comprises a sequencing reaction for example but not limited to, cycle sequencing or SBE. In certain embodiments, terminators are those in which the nucleotide base is a purine, a 7-deaza-purine, a pyrimidine, or a nucleotide analog, and the sugar moiety is a pentose which includes a 3′-substituent that blocks further synthesis, such as a dideoxynucleoside triphosphate (ddNTP). In certain embodiments, substituents that block further synthesis include, but are not limited to, amino, deoxy, halogen, alkoxy and aryloxy groups. Some non-limiting examples of terminators include, those in which the sugar-phosphate ester moiety is 3′-(C1-C6)alkylribose-5′-triphosphate; 2′-deoxy-3′-(C1-C6)alkylribose-5′-triphosphate; 2′-deoxy-3′-(C1-C6)alkoxyribose-5-triphosphate; 2′-deoxy-3′-(C5-C14)aryloxyribose-5′-triphosphate; 2′-deoxy-3′-haloribose-5′-triphosphate; 2′-deoxy-3′-aminoribose-5′-triphosphate; 2′,3′-dideoxyribose-5′-triphosphate; or 2′,3′-didehydroribose-5′-triphosphate. Terminators also include “T” terminators, including ddTTP and dUTP, which incorporate opposite an adenine, or adenine analog, in a template; “A” terminators, including ddATP, which incorporate opposite a thymine, uracil, or an analog of thymine or uracil, in the template; “C” terminators, including ddCTP, which incorporate opposite a guanine or a guanine analog, in the template; and “G” terminators, including ddGTP and ddITP, which incorporate opposite a cytosine or a cytosine analog, in the template. In some embodiments, a nucleotide terminator comprises a reporter group, for example but not limited to, a fluorescent reporter group.

Certain embodiments of the disclosed methods comprise a microfluidics device for at least one of: sample preparation; an amplification reaction, including a sequencing reaction; a purifying step; and obtaining the sequence of at least part of an amplification product. A microfluidics device is reaction vessel comprising at least one microchannel, generally comprising an internal dimension of one millimeter or less. Microfluidics device typically employ very small reaction volumes, often on the order of one or a few microliters, nanoliters (nL), or picoliters (pL). Those in the art will appreciate that the size, shape, and composition of a microfluidics device is generally not a limitation of the current teachings. Rather, a variety of suitable microfluidics devices can be employed in performing one or more steps of the disclosed methods. Descriptions of exemplary microfluidics devices and uses thereof can be found in, among other places, Fiorini and Chiu, BioTechniques 38:429-46, 2005; Kelly and Woolley, Analyt. Chem. 77(5):96A-102A, 2005; Cheuk-Wai Kan et al., Electrophoresis 25:3564-88, 2004; and Yeun et al., Genome Res. 11:405-12, 2001.

Certain Exemplary Component Techniques

According to the instant teachings, gDNA may be obtained from any living, or once living, organism, including but not limited to a prokaryote or a eukaryote, for example but not limited to a plant and an animal, including a human; and including cells and organs obtained from a prokaryote, a plant, or an animal, for example but not limited to cultured cells and blood cells. In certain embodiments, the gDNA may be present in a double-stranded or single-stranded form.

A variety of sample preparation techniques are available for obtaining certain gDNA comprising target regions for use with certain disclosed methods and kits. When the gDNA is obtained through isolation from a biological matrix, certain isolation techniques may include (1) organic extraction followed by ethanol precipitation, e.g., using a phenol/chloroform organic reagent (see, e.g., Ausubel et al.), in certain embodiments, using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., BioTechniques 10(4): 506-513, 1991; and (3) salt-induced DNA precipitation methods (e.g., Miller et al., Nucl. Acids Res. 16(3): 9-10, 1988, such precipitation methods being typically referred to as “salting-out” methods. In certain embodiments, the gDNA isolation technique comprises an enzyme digestion step to help eliminate unwanted protein from the sample, for example but not limited to, digestion with proteinase K (see, e.g., U.S. patent application Ser. No. 09/724,613).

In certain embodiments, nucleic acids in a sample, including gDNA, may be subjected to a cleavage or fragmentation procedure, for example but not limited to, sonication, shear force, or restriction enzyme digestion. In certain embodiments, such cleavage fragments serve as templates for a subsequent amplifying step.

The terms “amplifying” and “amplification” are used in a broad sense and refer to any technique by which at least a part of a gDNA or an amplification product, is reproduced or copied (including the synthesis of a complementary strand), typically in a template-dependent manner, including, a broad range of techniques for amplifying nucleic acid sequences, either linearly or exponentially. Some non-limiting examples of amplification techniques include primer extension, including the polymerase chain reaction (PCR), RT-PCR, asynchronous PCR (A-PCR), and asymmetric PCR, strand displacement amplification (SDA), multiple displacement amplification (MDA), nucleic acid strand-based amplification (NASBA), rolling circle amplification (RCA), transcription-mediated amplification (TMA), transcription, and the like, including multiplex versions or combinations thereof. Descriptions of certain amplification techniques can be found in, among other places, Sambrook and Russell; Sambrook et al.; Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); McPherson and Moller, PCR The Basics, Bios Scientific Publishers, Oxford, U.K., 2000 (“McPherson”); Rapley, The Nucleic Acid Protocols Handbook (2000), Humana Press, Totowa, N.J. (“Rapley”); U.S. Pat. Nos. 6,027,998 and 6,511,810; PCT Publication Nos. WO 97/31256 and WO 01/92579; Ehrlich et al., Science 252:1643-50 (1991); Favis et al., Nature Biotechnology 18:561-64 (2000); and Rabenau et al., Infection 28:97-102 (2000). The term “amplification product” includes the nucleic acid sequences generated from any number of cycles of amplification reactions, including primer extension reactions and RNA transcription reactions, unless otherwise apparent from the context.

In certain embodiments, amplification techniques comprise at least one cycle of amplification, for example, but not limited to, the sequential procedures of: selectively hybridizing primers to target flanking regions or primer-binding sites (or complements of either, as appropriate) of the gDNA or amplification products from any number of cycles of an amplification reaction; synthesizing a strand of nucleotides in a template-dependent manner using a polymerase; and denaturing the resulting nucleic acid duplex to separate the strands. The cycle may or may not be repeated. In certain embodiments, amplification techniques comprising transcription comprise at least one cycle of amplification including the sequential procedures of: interaction of a DNA-dependent RNA polymerase with a promoter sequence; synthesizing a strand of nucleotides in a template-dependent manner using the polymerase; and denaturing the resulting nucleic acid duplex to separate the strands. The cycle may or may not be repeated.

Amplification can comprise thermocycling or can be performed isothermally. In some embodiments, amplifying comprises a thermocycler, for example but not limited to a GeneAmp® PCR System 9700, 9600, 2700, or 2400 thermocycler (all from Applied Biosystems). In some embodiments, double-stranded amplification products are not initially denatured, but are used in their double-stranded form in one or more subsequent steps. In certain embodiments, single-stranded amplicons are generated in an amplification reaction, for example but not limited to asymmetric PCR or A-PCR.

Primer extension according to the present teachings is an amplification process comprising elongating a primer that is annealed to a template in the 5′ to 3′ direction using a template-dependent polymerase. According to certain embodiments, with appropriate buffers, salts, pH, temperature, and NTPs, including analogs and derivatives thereof, a template-dependent polymerase incorporates nucleotides complementary to the template strand starting at the 3′-end of an annealed primer, to generate a complementary strand. In certain embodiments, the polymerase used for primer extension lacks or substantially lacks 5′-exonuclease activity. Descriptions of certain primer extension reactions can be found in, among other places, Sambrook et al., Sambrook and Russell, and Ausubel et al.

Transcription according to certain embodiments is an amplification process comprising a DNA-dependent RNA polymerase interacting with a DNA promoter on a single- or double-stranded template and generating, in a 5′ to 3′ direction, an amplification product comprising a complementary strand of ribonucleotides. In certain embodiments, an amplification composition further comprises transcription factors. According to some embodiments, DNA-dependent RNA polymerases, including but not limited to T3, T7, and SP6 RNA polymerases, can interact with a promoter comprising a promoter sequence annealed with its complement. In some embodiments, a promoter sequence comprises a multiplicity of different sequences suitable for binding a DNA-dependent RNA polymerase, for example but not limited to a first sequence suitable for binding a first DNA-dependent RNA polymerase and a second sequence suitable for binding a second DNA-dependent RNA polymerase. Those in the art understand that as an amplification product is amplified by certain amplification means, the complement of the promoter sequence is synthesized in the complementary amplicon and collectively the promoter sequence and its complement form a double-stranded promoter suitable for binding certain polymerases. Thus, it is to be understood that the complement of a promoter sequence is expressly included within the intended meaning of the term promoter sequence, unless stated otherwise. The promoter sequence and its complement will be of sufficient length to permit an appropriate polymerase to interact with it. In some embodiments, an amplification product comprises an DNA-dependent RNA polymerase terminator sequence, a restriction enzyme site to facilitate run-off transcripts, or both. Descriptions of transcription and promoter sequences, including examples thereof, can be found in, among other places, Sambrook and Russell; Ausubel et al.; Lehninger, particularly in Chapter 26; Enzyme Resource Guide: Polymerases, Promega, Corporation, Madison, Wis., 1998; and The Basics: In Vitro Transcription, Ambion, Inc., 2005.

In certain embodiments, an amplification reaction comprises multiplex amplification, in which different target sequences or different amplification product species are simultaneously amplified using a multiplicity of different primer sets (see, e.g., Henegariu et al., BioTechniques 23:504-11, 1997; and Rapley, particularly in Chapter 79).

In certain embodiments, an amplifying reaction comprises asymmetric PCR. According to certain embodiments, asymmetric PCR comprises an amplification composition comprising (i) at least one primer set in which there is an excess of one primer, relative to the corresponding primer of the primer set, for example but not limited to a five-fold, a ten-fold, or a twenty-fold excess; (ii) at least one primer set that comprises only a forward primer or only a reverse primer; (iii) at least one primer set that, during given amplification conditions, comprises a primer that results in amplification of one strand and a corresponding primer that is disabled; or (iv) at least one primer set that meets the description of both (i) and (iii) above. Consequently, when the gDNA target region or an amplification product is amplified, an excess of one strand of the subsequent amplification product (relative to its complement) is generated. Descriptions of asymmetric PCR, can be found in, among other places, McPherson, particularly in Chapter 5; and Rapley, particularly in Chapter 64.

In certain embodiments, one may use at least one primer set wherein the melting temperature (Tm₅₀) of one of the primers is higher than the Tm₅₀ of the other primer, sometimes referred to as A-PCR (see, e.g., Published U.S. Patent Application No. US 2003-0207266 A1). In certain embodiments, the Tm₅₀ of the forward primer is at least 4-15° C. different from the Tm₅₀ of the corresponding reverse primer. In certain embodiments, the Tm₅₀ of the forward primer is at least 8-15° C. different from the Tm₅₀ of the corresponding reverse primer. In certain embodiments, the Tm₅₀ of the forward primer is at least 10-15° C. different from the Tm₅₀ of the corresponding reverse primer. In certain embodiments, the Tm₅₀ of the forward primer is at least 10-12° C. different from the Tm₅₀ of the corresponding reverse primer. In certain embodiments, in at least one primer set, the Tm₅₀ of a forward primer differs from the melting temperature of the corresponding reverse primer by at least about 4° C., by at least about 8° C., by at least about 10° C., or by at least about 12° C.

In certain embodiments of A-PCR, in addition to the difference in Tm₅₀ of the primers in a primer set, there is also an excess of one primer relative to the other primer in the primer set. In certain embodiments, there is a five- to twenty-fold excess of one primer relative to the other primer in the primer set. In certain embodiments of A-PCR, the primer concentration is at least 50 nM.

In A-PCR according to certain embodiments, one may use conventional PCR in the first cycles such that both primers anneal and both strands are amplified. By raising the temperature in subsequent cycles, however, one may disable the primer with the lower Tₘ such that only one strand is amplified. Thus, the subsequent cycles of A-PCR in which the primer with the lower Tₘ is disabled result in asymmetric amplification. Consequently, when the target region or an amplification product is amplified, an excess of one strand of the subsequent amplification product (relative to its complement) is generated.

According to certain embodiments of A-PCR, the level of amplification can be controlled by changing the number of cycles during the first phase of conventional PCR cycling. In such embodiments, by changing the number of initial conventional cycles, one may vary the amount of the double-stranded amplification products that are subjected to the subsequent cycles of PCR at the higher temperature in which the primer with the lower Tₘ is disabled.

In some embodiments, an amplification reaction is followed by a “clean-up” or “purifying” step, wherein at least some of the components of the amplification composition are removed from at least some of the amplification products, thereby purifying the amplification products. Purifying typically comprises a degrading means, including an enzyme such as a nuclease or a phosphatase, or a separating means, including a physical separation means such as a spin column or a separation based on hybridization, such as hybridization-based pullout. For example but not limited to degrading and/or separating at least some of the unincorporated primers, unincorporated NTPs, including in some embodiments nucleotide terminators, extending enzymes, salts, other amplification composition components, or combinations thereof. In some embodiments, purifying an amplification product comprises a “spin column” or other centrifugal or gel-based separation means; a degradation reaction comprising for example an exonuclease, a phosphatase, or both (e.g., ExoSAP-It® reagent), or an exonuclease and an apyrase; a hybridization-based separation means; or a precipitation step, for example but not limited to ethanol precipitation in the presence of a salt, such as sodium or potassium acetate.

The term “degrading” is used in a broad sense herein and refers to any technique in which: (i) an unincorporated NTP, including in some embodiments a nucleotide terminator, is rendered unincorporable, typically by enzymatic digestion by a phosphatase, (ii) an unincorporated primer is digested, typically by an exonuclease, (iii) at least one nucleotide is removed from a polynucleotide or in which at least one internucleotide bond in a polynucleotide is cleaved, including chemical means such as alkaline hydrolysis and enzymatic means for example but not limited to treatment by a nuclease, or (iv) combinations thereof.

In some embodiments, purifying comprises a nuclease, such as a DNase or an RNase, for example but not limited to exonuclease I, mung bean nuclease, S1 nuclease, exonuclease T, RNase H, RNase A, RNase I, RNase III, or combinations thereof. In some embodiments, a NTP and/or an unincorporated primer is degraded. In some embodiments, one strand of a double-stranded amplification product is degraded, for example but not limited to, an RNA strand annealed with a complementary DNA strand is degraded by, for example but not limited to RNase H, RNase A, or alkaline hydrolysis. In some embodiments, unincorporated NTPs are degraded using an apyrase or a phosphatase, including shrimp alkaline phosphatase (SAP) or calf intestinal phosphatase (CIP). In some embodiments, degrading unincorporated primers and unincorporated NTPs comprises an apyrase, an inorganic pyrophosphate (PPi), and an exonuclease I. Those in the art will appreciate that the method for degrading unincorporated primers and/or unincorporated NTPs is typically not limiting, provided that the desired polynucleotides, typically amplification products (or in some embodiments, one strand of a double-stranded amplification product), are not degraded or at least not substantially degraded, while the unincorporated primers and NTPs are degraded.

In some embodiments, unincorporated primers, unincorporated NTPs, amplification composition reagents, or combinations thereof, are separated from an amplification product by, for example but not limited to, gel or column purification, sedimentation, filtration, beads, including streptavidin-coated beads, magnetic separation, or hybridization-based pull out, including annealing amplification products comprising hybridization tags to a capture surface. A number of kits and reagents for performing such separation techniques are commercially available, including the Wizard® MagneSil™ PCR Clean-Up System (Promega), the MinElute PCR Purification Kit, the QIAquick Gel Extraction Kit, the QIAquick Nucleotide Removal Kit, the QIAquick 96 PCR Purification Kit or BioRobot Kit (all from Qiagen, Valencia, Calif.), Dynabeads® (Dynal Biotech), or the ABI PRISM® Duplex™ 384 Well F/R Sequence Capture Kit (Applied Biosystems P/N 4308082). In some embodiments, an amplification product is not purified prior to an amplifying reaction, including certain sequencing techniques.

The term “sequencing” is used in a broad sense herein and refers to any technique known in the art that allows the order of at least some consecutive deoxyribonucleotides in at least part of an amplification product to be obtained and from which at least part of the sequence of the gDNA target region is determined. Some non-limiting examples of sequencing techniques include Sanger's dideoxy termination method and the chemical cleavage method of Maxam and Gilbert, including variations of those methods; sequencing by hybridization; sequencing by synthesis; and restriction mapping. In certain embodiments, sequencing comprises electrophoresis, including gel electrophoresis and capillary electrophoresis, including miniaturized capillary electrophoresis, and often comprising laser-induced fluorescence; sequencing by hybridization including bead array microarray hybridization; microfluidics (see, e.g., Paegel et al., Analyt. Chem. 74:5092-98, 2002); mass spectrometry (see, e.g., Köster et al., Nat. Biotechnol. 14:1123-28, 1996); single molecule detection, including fluorescence microscopy or a nanometer-scale pore or nanopore; or combinations thereof. In some embodiments, sequencing comprises direct sequencing, duplex sequencing, cycle sequencing, single base extension (SBE) sequencing, solid-phase sequencing, Simultaneous Bi-directional Sequencing (SBS), double ended sequencing (see, e.g., Published PCT Application No. WO 2004/070005 A2), or combinations thereof. In some embodiments, sequencing comprises asymmetric PCR or A-PCR. In some embodiments, sequencing comprises an extending enzyme comprising a first fluorescent reporter group, such as a FRET donor, and a NTP comprising a second fluorescent reporter group, such as a quencher (see, e.g., U.S. Published Patent Application No. US 2003/0064366 A1). In some embodiments, sequencing comprises detecting at least some amplification products using an instrument, for example but not limited to an ABI PRISM® 377 DNA Sequencer, an ABI PRISM® 310, 3100, 3100-Avant, 3730, or 3730xI Genetic Analyzer, an ABI PRISM® 3700 DNA Analyzer (all from Applied Biosystems), a microarray or bead array, a fluorimeter, or a mass spectrometer. In some embodiments, sequencing comprises incorporating a dNTP, including a dATP, a dCTP, a dGTP, a dTTP, a dUTP, a dITP, or combinations thereof and including dideoxyribonucleotide versions of dNTPs (e.g., ddATP, ddCTP, ddGTP, ddITP, ddTTP, and ddUTP), into an amplification product. In some embodiments, sequencing comprises a sequencing grade DNA-dependent DNA polymerase, for example but not limited to, AmpliTaq DNA polymerase CS or FS (Applied Biosystems); Sequenase or Thermo Sequenase (USB Corp.); and Sequencing Grade Taq DNA Polymerase (Promega). In some embodiments, sequencing comprises: a DNA-dependent DNA polymerase, for example but not limited to the Klenow fragment of E. coli DNA Pol I; an ATP sulfurylase, for example but not limited to a recombinant S. cerevisiae ATP sulfurylase, a luciferase, including firefly luciferase, or a sulfurylase-luciferase fusion protein (a non-limiting example of an enzymatically active mutant or variant of an ATP sulfurylase and of a luciferase; see, e.g., U.S. Patent Publication Nos. US 2003/0113747 A1 and US 2003/0119012 A1); and optionally, an apyrase. In some embodiments, a sequencing reaction comprises dATPαS, typically in place of dATP. In some embodiments, sequencing further comprises detecting light or fluorescence using, for example but not limited to a photodiode, a photomultiplier tube, a charge-coupled camera (CCD), a fluorimeter, a laser-scanner coupled with a detector, or combinations thereof.

Those in the art will appreciate that the sequencing method employed is not typically a limitation of the disclosed methods. Rather any sequencing technique that provides the order of at least some consecutive deoxyribonucleotides of at least part of an amplification product can typically be used with the current methods. Descriptions of exemplary sequencing techniques can be found in, among other places, McPherson, particularly in Chapter 5; Sambrook and Russell; Ausubel et al.; Siuzdak, The Expanding Role of Mass Spectrometry in Biotechnology, MCC Press, 2003, particularly in Chapter 7; Di Giusto and King, Nucl. Acids Res. 31 :e7; Schena, Microarray Analysis, John Wiley & Sons, 2003, particularly in Chapter 13; BigDye® Terminator v 1.1 or v3.1 Cycle Sequencing Kit Protocols (Applied Biosystems P/N 4337036 or 4337035, respectively); Ronaghi, Genome Res. 11:3-11, 2001; Agah et al., Nucl. Acids Res. 32:e166, 2004; Kartalov and Quake, Nucl. Acids Res. 32:2873-79, 2004; Cheuk-Wai Kan et al., Electrophoresis 25:3564-88, 2004; and Rapley.

In some embodiments, the amplification products of a sequencing reaction are purified before obtaining the sequence of the sequencing reaction products by enzymatic degradation, including exonuclease I and SAP digestion, for example but not limited to the ExoSAP-IT® reagent (USB Corporation). In some embodiments, purifying the sequencing reaction products comprises a separation means, including gel or column purification, sedimentation, filtration, beads, magnetic separation, or hybridization-based pull out (see, e.g., ABI PRISM® DupleX™ 384 Well F/R Sequence Capture Kit, Applied Biosystems P/N 4308082).

Exemplary Embodiments

Methods and kits are disclosed for amplifying and sequencing gDNA target regions. Those in the art will appreciate that the current teachings obviate, at least in some applications, the need for cloning gDNA target sequences into vectors for in vivo amplification in appropriate host cells to generate sufficient starting material for sequencing and evaluation. The disclosed methods comprise a multiplicity of amplification reactions, each comprising an extending enzyme, for example but not limited to an RNA-dependent DNA polymerase, a DNA-dependent DNA polymerase, a DNA-dependent RNA polymerase, an RNA-dependent RNA polymerase, or combinations thereof. According to some embodiments, an amplifying reaction comprises: PCR or at least primer extension, transcription, RT-PCR or reverse transcription followed by PCR, or combinations thereof. Certain sequencing techniques comprise an amplification reaction, for example but not limited to, cycle sequencing, SBE, and pyrosequencing.

Certain disclosed methods comprise at least two different PCR reactions, including a first PCR amplifying reaction comprising a limited number of cycles of amplification, for example but not limited to about 5-15 cycles, 8 cycles, 10 cycles, 12 cycles, or 15 cycles, that typically is performed in multiplex (sometimes referred to as a pre-amplification or Booster Amp step, see, e.g., U.S. Pat. No. 6,605,451; U.S. Patent Application Publication No. US 2004/0175733A1) to generate a first amplification product or a multiplicity of different first amplification products; and a “conventional” PCR reaction, typically comprising 20-40 cycles or more, 25 cycles, 30 cycles, 35 cycles, 40 cycles, 45 cycles, or 50 cycles, and typically performed as a single-plex reaction, including a multiplicity of massively parallel single-plex reactions to generate a second amplification product. In some embodiments, the second amplification product is sequenced. In some embodiments, a first PCR reaction is used to amplify a target region and a subsequent PCR reaction is performed after a RT reaction or in conjunction with an RT reaction, i.e., an RT-PCR reaction.

In some embodiments, a first amplification composition comprising a multiplicity of different first amplification products is diluted in a suitable diluent, including, nuclease-free water or an appropriate buffer (for example but not limited to 1:5, 1:10, 1:15, or 1:20, first amplification composition:diluent). In some embodiments, a second amplification composition comprises at least some of the “diluted” first amplification composition comprising first amplification products, including an aliquot or portion of the diluted first amplification composition. In some embodiments, a multiplicity of different second amplification compositions each comprise some of the diluted first amplification composition, for example, an equal portion of the diluted first amplification product.

According to the certain disclosed methods, a “clean-up” step is performed after one or more amplification reaction to degrade and/or remove unincorporated primers, unincorporated nucleotide triphosphates (NTPs), amplification composition reagents, or combinations thereof, using techniques known in the art. In some embodiments, an amplification composition comprising amplification products is degraded using a degrading means, including an enzymatic degrading means, for example but not limited to a nuclease and/or a phosphatase, or a chemical degrading means such as alkaline hydrolysis using for example NaOH. In some embodiments, unincorporated primers, unincorporated NTPs, amplification composition reagents, or combinations thereof, are removed from an amplification composition comprising amplification products using a separating means, including a spin column, polymer, magnetic or para-magnetic beads, hybridization with “pull-out” sequences, or precipitation, for example but not limited to ethanol precipitation in the presence of sodium acetate, potassium acetate, or other appropriate salt. A “purified” amplification product is obtained from such clean-up steps. Those in the art appreciate that in certain methods a clean-up step(s) may be desirable after one or more amplifying reaction. In some embodiments, the RNA component of an RNA:cDNA amplification product is degraded using a degrading means such as RNase H or alkaline hydrolysis.

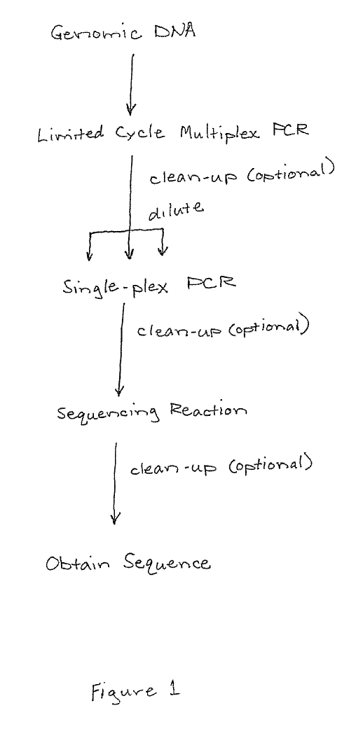

In one illustrative embodiment, depicted in FIG. 1, a small amount of nucleic acid, e.g., 1 ng of gDNA, is amplified in a first amplification composition comprising a first extending enzyme and a multiplicity of different target-specific primer sets, for example but not limited to a multiplex PCR pre-amplification reaction, to generate a multiplicity of different first amplification products. The first reaction composition comprising the multiplicity of different first reaction products is diluted in nuclease-free water. In some embodiments, the first amplification products or the diluted first amplification products are purified using a clean-up step before the second amplifying reaction. At least some of the diluted first amplification products are added to a second amplification composition comprising a second extending enzyme, dNTPs, and a second amplification product primer set. Under suitable conditions, a second amplification product is generated. In some embodiments, an aliquot of the first amplification products or an aliquot of the purified first amplification products is combined with each of a multiplicity of different second amplification compositions each comprising a different second amplification product primer set. In some embodiments, a multiplicity of different second amplification products are generated, each in a different second amplification composition, during a massively parallel second amplification reaction. In some embodiments, a massively parallel amplifying step comprises a multi-well reaction vessel, including a plate comprising multiple reaction wells, for example but not limited to, a 24-well plate, 96-well plate, a 384-well plate, or a 1536-well plate; or a multi-chamber microfluidics device, for example but not limited to a TaqMan Low Density Array wherein each chamber comprises an appropriate primer set (Applied Biosystems).

The nucleotide sequence of at least a part of the second amplification product is obtained and the corresponding sequence of the gDNA target region is determined. In some embodiments, sequencing at least part of a second amplification product comprises forming a third amplification composition or a multiplicity of third amplification compositions comprising at least some of the second amplification products or at least some of the purified second amplification products, a DNA-dependent DNA polymerase, a sequencing primer or a pair of sequencing primers, a nucleotide terminator or a dNTP and a nucleotide terminator; and amplifying the second amplification product in the third amplification composition(s) to generate a third amplification product, for example but not limited to a series of termination products. The nucleotide sequence of at least part of the third amplification product is obtained and the corresponding sequence of the gDNA target region is determined. In some embodiments, the third amplification products are purified before obtaining at least part of their sequence.

In some embodiments, at least one sequencing primer comprises a reporter group and at least one reporter group-labeled amplification product is generated. In some embodiments, at least one nucleotide terminator, for example but not limited to a ddNTP, comprises a reporter group and a reporter group-labeled amplification product is generated. The nucleotide sequence of at least part of the reporter group-labeled amplification product is obtained and the corresponding sequence of the gDNA target region is determined. In some embodiments, the reporter group-labeled amplification product is purified before obtaining at least some of its nucleotide sequence. In some embodiments, the sequencing comprises resequencing. In some embodiments, sequencing comprises sequencing by hybridization, sequencing by synthesis, chemical cleavage, restriction mapping, mass spectroscopy, a microfluidics device, capillary electrophoresis, or combinations thereof.

In certain exemplary embodiments, air-dried amplification product pellets, comprising amplification products, including sequencing reaction products, and/or amplification products of uniquely identifiable molecular weight, are resuspended in buffer or deionized formamide, e.g., HiDi formamide (Applied Biosystems). In certain embodiments, the resuspended samples and a molecular weight marker (e.g., GS 500 size standard, Applied Biosystems, Foster City, Calif.) are loaded onto an electrophoresis platform (e.g., ABI PRISM™ Genetic Analyzer, Applied Biosystems) and electrophoresed in an appropriate polymer, for example but not limited to, POP-4, POP-6, or POP-7 polymers (Applied Biosystems). In certain embodiments, the electrophoretic bands comprising at least some of the sequencing products are detected and their nucleotide sequence is obtained. In certain embodiments, the bands are identified based on their relative electrophoretic mobility and the corresponding nucleotide sequence is obtained.

In some embodiments, the disclosed methods comprise a microfluidics device, “lab on a chip”, or micrototal analytical system (μTAS). In some embodiments, sample preparation is performed in a microfluidics device. In some embodiments, an amplification reaction is performed in a microfluidics device. In some embodiments, a sequencing reaction is performed in a microfluidic device. In some embodiments, the nucleotide sequence of at least a part of an amplification product is obtained using a microfluidics device. Descriptions of exemplary microfluidic devices can be found in, among other places, Published PCT Application Nos. WO/0185341 and WO 04/011666; Kartalov and Quake, Nucl. Acids Res. 32:2873-79, 2004; and Fiorini and Chiu, BioTechniques 38:429-46, 2005.

According to certain disclosed methods, a gDNA target region is amplified in a first amplification composition comprising a target-specific primer set and a first amplification product is generated. At least one of the primers of the target-specific primer set comprises a tail portion comprising a promoter sequence or the complement of a promoter sequence and at least one strand of the first amplification product comprises the promoter sequence or the complement of the promoter sequence. In some embodiments, the first amplification product is purified. A second amplification composition is formed comprising at least some of the first amplification product comprising the promoter sequence or at least some of the purified first amplification products comprising the promoter sequence or its complement, a DNA-dependent RNA polymerase, and rNTPs; and under suitable conditions, a second amplification product comprising ribonucleotides is generated. In some embodiments, the second amplification product is purified. A third amplification composition is formed comprising at least some of the second amplification products or at least some of the purified second amplification products, a third amplification primer set or at least a third amplification primer, an RNA-dependent DNA polymerase or a DNA-dependent DNA polymerase capable of reverse transcription, and dNTPs. Under suitable conditions, a third amplification product is generated. The third amplification product is contacted with a third amplification product primer and, under suitable conditions, a fourth amplification product is generated. In some embodiments, the third amplification composition further comprises a DNA-dependent DNA polymerase, for example but not limited to a “hot start” DNA polymerase, and a one-step RT-PCR reaction can occur. In other embodiments, the DNA-dependent DNA polymerase is added to the third amplification composition after the third amplification product is generated and the amplifying comprises a two-step RT-PCR reaction. In some embodiments, at least part of the fourth amplification product is sequenced.

In some embodiments, sequencing at least part of the fourth amplification product comprises forming a fourth amplification composition or a multiplicity of fourth reaction compositions comprising at least some of the fourth amplification product, a DNA-dependent DNA polymerase, a sequencing primer or a sequencing primer set, a nucleotide terminator or a dNTP and a nucleotide terminator; and amplifying the fourth amplification product in the fourth amplification composition(s) to generate a fifth amplification product. In some embodiments, the fifth amplification product is purified. The nucleotide sequence of at least part of the fifth amplification product or at least part of the purified fifth amplification product is obtained and the corresponding sequence of the gDNA target region is determined.

In some embodiments, at least one sequencing primer comprises a reporter group and a reporter group-labeled amplification product is generated. In some embodiments, at least one nucleotide terminator comprises a reporter group and at least one reporter group-labeled amplification product is generated. In some embodiments, the reporter group-labeled amplification product is purified before obtaining at least some of its nucleotide sequence. The nucleotide sequence of at least part of the reporter group-labeled amplification product or at least part of the purified reporter group-labeled amplification product is obtained and the corresponding sequence of the gDNA target region is determined.

In some embodiments, a target-specific primer set that comprises a forward target-specific primer, a corresponding second forward primer, and a corresponding reverse target-specific primer, and the first amplifying reaction comprises a two stage process. Typically, the forward target-specific primer is incorporated into the first stage first amplification products during the first stage and the second forward primer is incorporated into corresponding second stage amplification products during the second stage of the first amplification reaction. In some embodiments, all three primers of the target-specific primer set(s) are initially present in the first amplification composition. In some embodiments, the initial first amplification composition comprises the forward target-specific primer and the corresponding reverse target-specific primer of the target-specific primer set(s), but not the second forward primer; and the second forward primer is added to the first amplification composition after at least one amplification cycle has been performed to generate at least some first stage first amplification product that comprise the sequence of the forward target-specific primer or its complement.

In some embodiments, particularly when a primer set comprised at least one tailed primer comprising a promoter sequence was incorporated into an amplification product, a subsequent amplifying reaction can comprise in vitro transcription (IVT). For example but without limitation, an amplification composition is formed comprising at least some of the amplification products comprising the promoter sequence or at least some of the purified amplification products comprising the promoter sequence, a DNA-dependent RNA polymerase, and rNTPs; and, under appropriate conditions, a multiplicity of amplification products comprising ribonucleotide are generated, e.g., cRNA. In some embodiments, the amplification products comprising ribonucleotides are purified by digesting the amplification composition comprising the ribonucleotide amplification products or by separating the ribonucleotide amplification products from at least some of the reaction components of the amplification composition. In some embodiments, an amplification composition is formed comprising at least some of the amplification product comprising ribonucleotide polymers or at least some of the purified amplification products comprising ribonucleotide polymers, an extending enzyme, for example but not limited to a RNA-dependent DNA polymerase or a DNA-dependent DNA polymerase that possesses reverse transcriptase activity, and dNTPs; and under suitable reaction conditions, an amplification product comprising the ribonucleotide amplification product duplexed with a complementary DNA (cDNA) is generated. In some embodiments, the cRNA:cDNA amplification product is degraded to remove the RNA amplification product. In some embodiments, the amplification composition further comprises a DNA-dependent DNA polymerase and/or primer sets; or a DNA-dependent DNA polymerase and/or primer sets is added to the amplification composition comprising the cDNA amplification product. Under suitable reaction conditions, for example but not limited to, thermocycling, a double-stranded DNA amplification product is generated, for example by PCR or RT-PCR, as appropriate. In some embodiments, PCR comprises asymmetric PCR or A-PCR and single-stranded and double-stranded DNA amplification products are generated.

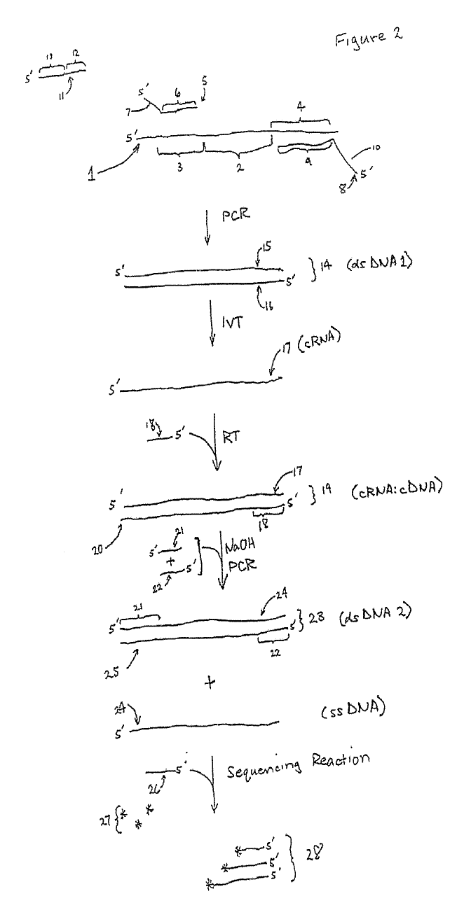

An exemplary method for determining the nucleotide sequence of at least one gDNA target region comprising five amplifying reactions, a first PCR, an IVT reaction, an RT reaction, a second PCR, and a sequencing reaction comprising primer extension, is schematically depicted in FIG. 2. A first amplification composition is formed, comprising a first extending enzyme, a very small amount of single-stranded gDNA (1) comprising a target region (2), an upstream target flanking region (3) and a downstream target flanking region (4), and a target-specific primer set comprising: a forward target-specific primer (5) comprising a first target-binding portion (6) and a first tail portion (7) comprising a first primer-binding site; a reverse target-specific primer (8) comprising a second target-binding portion (9) and a second tail portion (10) comprising a second primer-binding portion; and a second forward primer (11) comprising a sequence (12) designed to anneal with the first primer-binding portion (7) of the incorporated forward target-specific primer and a promoter sequence or its complement (13).

In some embodiments, all three primers of the first primer set are initially included in the first amplification composition. In other embodiments, the first amplification composition comprises a forward target-specific primer but not a second forward primer and under suitable conditions, a first stage first amplification product comprising the sequence of the first target-specific primer or its complement is generated. The second forward primer is then added to the amplification composition and its sequence or the complement of the second forward primer sequence is incorporated into second stage amplification products. The first amplification composition is subjected to an amplification reaction comprising PCR and a double-stranded first amplification product (14), depicted as “dsDNA 1” in FIG. 2, is generated comprising a first strand (15) and a second strand (16), each comprising the promoter sequence or its complement.

In some embodiments, wherein for example the forward target-specific primer and the second forward primer are added to the first amplification composition separately, a “clean-up” or purifying step is performed on the first amplification composition comprising the first stage first amplification products including incorporated first forward primers or a sequence that is complementary to a first forward primer. In some embodiments, the purifying comprises degrading using an exonuclease to remove any unincorporated first forward primers, then the exonuclease is denatured prior to adding the second forward primers. In some embodiments, the purifying comprises removing the unincorporated first forward primers using a separating means such as a spin column before adding the second forward primers. According to certain embodiments, in the second stage of the first amplification reaction, the second forward primers are incorporated into the amplification products. Thus, in this illustrative embodiment, the final amplification products (14) of the first amplification reaction comprise the sequence of the forward target-specific primer, the sequence of the second forward primer including the promoter sequence, and the sequence of the reverse target-specific primer, including or alternatively sequences complementary to any of these, in the first strand (15) and/or the second strand (16) of the double-stranded first amplification product (14).

An illustrative second amplification composition is formed, comprising at least some of the first amplification product (14) or at least one strand of the amplification product comprising the promoter sequence (16), an appropriate DNA-dependent RNA polymerase, and rNTPs. The second amplification composition is subjected to at least one amplification cycle comprising in vitro transcription, shown as “IVT” in FIG. 2, and a second amplification product (17) is generated, shown as “cRNA” in FIG. 2. In some embodiments, the second amplification product is purified.

An illustrative third amplification composition is formed, comprising at least some of the second amplification product (17) or at least some of the purified second amplification product, a reverse transcriptase, a reverse primer (18), and dNTPs. The third amplification composition is subjected to at least one amplification cycle comprising reverse transcription (shown as “RT”) and a third amplification product (19), comprising a cRNA strand (17) duplexed with a cDNA strand (20), shown as “cRNA:cDNA” in FIG. 2, is generated. Sodium hydroxide, shown as “NaOH” in FIG. 2, is added to the third amplification composition to degrade the cRNA strand by alkaline hydrolysis and then the third reaction composition is neutralized. In other embodiments, degrading the cRNA comprises nuclease digestion, for example but not limited to treatment with RNase H.

An illustrative fourth amplification composition is formed, comprising the neutralized third amplification composition comprising the single-stranded cDNA, an extending enzyme, in this example, a DNA-dependent DNA polymerase, and third amplification product primer set comprising a forward primer (21) and a reverse primer (22), wherein the relative concentration of the forward primer (21) is in excess of the reverse primer (22), for example but not limited to a 10-fold or 20-fold excess. The fourth amplification composition is subjected to a multiplicity of amplification cycles comprising asymmetric PCR to generate a double-stranded fourth amplification product (23) comprising a first strand (24) comprising the sequence of the forward fourth amplification product primer and a second strand (25) comprising the sequence of the reverse fourth amplification product primer, shown as “dsDNA 2”, and a multiplicity of single-stranded fourth amplification products (24), shown as “ssDNA” in FIG. 2.

In some embodiments, an amplification composition does not comprise an excess of one primer and the subsequent amplification reaction comprises conventional PCR. In some embodiments, one of the primers of the corresponding amplification product primer set comprises an affinity tag, for illustration purposes but not as a limitation, a biotin moiety, which is incorporated into one strand of the subsequent double-stranded amplification product. The double-stranded amplification product comprising one biotinylated strand is combined with a streptavidin-coated capture surface, for example but not limited to magnetic or polymer beads, wells of a microtiter plate, or a glass slide, and the affinity partners bind. The exemplary biotinylated double-stranded amplification product, bound to the capture surface by the avidin-streptavidin bond, is denatured, releasing the non-biotinylated strand. Subsequently, the released strand, the bound strand, or both can be used for a subsequent amplifying reaction and/or sequencing.

Returning to FIG. 2, an illustrative fifth amplification composition is formed, comprising at least some of the single-stranded fourth amplification products (24), a sequencing primer (26), an extending enzyme, in this illustration a sequencing grade DNA-dependent DNA polymerase, for example but not limited to, AmpliTaq DNA polymerase CS, Thermo Sequenase, or TopoTaq FS, and dNTPs (27), including reporter group-labeled nucleotide terminators, shown as “*” in FIG. 2. The fifth amplification composition is subjected to at least one amplification cycle comprising cycle sequencing and a multiplicity of different fifth amplification products (28) are generated, in this example, a family of reporter group-labeled termination fragments.

In some embodiments, a sequencing reaction comprises a multiplicity of different amplification compositions, for example but not limited to, four different amplification compositions, each comprising a different reporter group-labeled nucleotide terminator, such as ddATP, ddCTP, ddGTP, and ddTTP; or four different amplification compositions, each comprising a different reporter group-labeled primer and an unlabeled nucleotide terminator, for example wherein each of the different reporter group-labeled primers corresponds to one unlabeled ddNTP. The nucleotide sequence of the family of reporter group-labeled termination fragments is obtained using, for example but not limited to, capillary electrophoresis and laser-induced fluorescence, and the sequence of the gDNA target region is determined. In other embodiments, a sequencing reaction comprises the Klenow fragment of E. coli DNA Pol I, an ATP sulfurylase, for example but not limited to a recombinant S. cerevisiae ATP sulfurylase, a luciferase, for example but not limited to, firefly luciferase, or a sulfurylase-luciferase fusion protein (a non-limiting example of an enzymatically active mutant or variant of an ATP sulfurylase and of a luciferase; see, e.g., U.S. Patent Publication Nos. US 2003/0113747 A1 and US 2003/0119012 A1), and optionally, an apyrase. In such embodiments, sequencing further comprises detecting light emitted from the sequencing reaction composition as each nucleotide is incorporated using, for example but not limited to a photodiode, a photomultiplier tube, or a charge-coupled camera (CCD).

Those in the art will appreciate that depending on the sequencing technique employed, a sequencing reaction may not be needed and that either a double-stranded amplification product (e.g., 23) can be sequenced, for example but not limited to by a chemical cleavage technique; or the nucleotide sequence of a released single strand (e.g., 24 and/or 25) of a double-stranded amplification product or a single-stranded amplification product generated by, for example, asymmetric PCR or A-PCR, can be obtained using, for example but not limited to, sequencing by hybridization or a mass spectrometer.

Exemplary Kits

The instant teachings also provide kits designed to expedite performing the subject methods. Kits typically serve to expedite the performance of the disclosed methods by assembling two or more components required for carrying out the methods. In some embodiments, kits contain components in pre-measured unit amounts to minimize the need for measurements by end-users. In some embodiments, kits include instructions for performing one or more of the disclosed methods. The kit components are typically optimized to operate in conjunction with one another.

In some embodiments, kits for determining the sequence of a gDNA target region comprise a first DNA-dependent DNA polymerase, a second DNA-dependent DNA polymerase, a DNA-dependent RNA polymerase, an RNA-dependent DNA polymerase, and at least one primer set or at least a primer. In some embodiments, the second DNA-dependent DNA polymerase and the RNA-dependent DNA polymerase comprise the same polymerase, e.g., a DNA-dependent DNA polymerase capable of reverse transcription or a reverse transcriptase capable of using a DNA template, for example but not limited to, Thermus thermophilus (Tth) DNA polymerase, AMV reverse transcriptase or MMLV reverse transcriptase.

Some kit embodiments comprise a target-specific primer set for each gDNA target region to be sequenced, wherein each target specific primer set comprises (a) a forward target-specific primer comprising (i) a first target-binding portion that comprises a sequence that is the same as or substantially the same as a first target flanking region and (ii) an upstream tail portion comprising a first primer-binding site, a first promoter sequence, or a first primer-binding site and a first promoter sequence and (b) a corresponding reverse target-specific primer comprising (i) a second target-binding portion that comprises a sequence that is complementary to or substantially complementary to a corresponding second target flanking region and (ii) an upstream tail portion comprising a second primer-binding site, a second promoter sequence, or a second primer-binding site and a second promoter sequence. In some embodiments, a target-specific primer set comprises (a) a forward target-specific primer comprising (i) a first target-binding portion that comprises a sequence that is the same as or substantially the same as a first target flanking region and (ii) an upstream tail portion comprising a first primer-binding site; (b) a corresponding reverse target-specific primer comprising (i) a second target-binding portion that comprises a sequence that is complementary to or substantially complementary to a corresponding second target flanking region and (ii) an upstream tail portion comprising a second primer-binding site; and (c) a second forward primer comprising (i) a sequence that is the same as or complementary with the first primer-binding site of the forward target-specific primer, the second primer-binding site of the reverse target-specific primer, or both and (ii) a promoter sequence. In some embodiments, the promoter sequence comprises a multiplicity of different promoter sequences, for example but not limited to a T3 DNA-dependent RNA polymerase promoter sequence, a T7 DNA-dependent RNA polymerase promoter sequence, and an SP6 DNA-dependent RNA polymerase promoter sequence.

In some embodiments, kits further comprise a multiplicity of primer sets for performing a multiplexed amplification reaction, for example but not limited to, a multiplexed pre-amplification reaction. In some embodiments, kits comprise 2-24 different primer sets, 25-96 different primer sets, 384 different primer sets, 1536 different primer sets, 6144 different primer sets, or greater than 6144 different primer sets.

In some embodiments, kits further comprise a third DNA-dependent DNA polymerase, for example but not limited to a sequencing grade polymerase, i.e., a DNA-dependent DNA polymerase with an enhanced ability to incorporate certain nucleotide terminators, such as ddNTPs. Non-limiting examples of sequencing polymerases include Therminator™ DNA polymerase (New England BioLabs, Beverly, Mass.), AmpliTaq DNA polymerase CS, AmpliTaq DNA polymerase FS (Applied Biosystems), Sequenase™, and Thermo Sequenase™ (USB Corp.)(see, e.g., Parker et al., BioTechniques 21:694-99, 1996; Vander Horn et al., BioTechniques 22:758-65, 1997). In some embodiments, a kit further comprises a sequencing primer or a pair of sequencing primers for priming DNA synthesis for sequencing reactions.

The current teachings, having been described above, may be better understood by reference to examples. The following examples are intended for illustration purposes only, and should not be construed as limiting the scope of the teachings herein in any way.

### EXAMPLE 1

### gDNA Amplification and Sequencing Method Comprising Two Amplification Reactions

This exemplary method combines (a) a multiplex PCR pre-amplification reaction comprising a mix of 24 target-specific primer sets and a small amount of human gDNA, (b) a multiplicity of different single-plex PCR reactions, and (c) a cycle sequencing reaction to amplify and resequence twenty-four target regions in each of four human gDNA samples.

Step 1: Multiplex PCR Pre-Amplification Reaction (First Amplification Reaction).

Twenty-four different resequencing amplicons (RSAs)(i.e., illustrative gDNA target regions), shown in Table 1, from four different human gDNA samples (Coriell Cell Repositories, Camden N.J., Repository #NA00893, NA10924, NA14529, and NA14672) were amplified using 24 corresponding target-specific primer sets, also shown in Table 1, in a limited cycle multiplex PCR (“pre-amplification”). All steps were performed on ice when possible, unless otherwise noted. Four parallel first amplification compositions were formed in each of four wells of a MicroAmp 96-well plate (Applied Biosystems, Foster City, Calif.), each comprising 5 μL AmpliTaq Gold® PCR Master Mix (Applied Biosystems P/N 4318739), 2.4 μL of the primer mix (containing 24 primer sets, 17 nM each primer), 1.6 μL glycerol (50%), and 1 μL of one of the four gDNAs (1 ng/μL). The plate was covered with MicroAmp Adhesive Film and an ABI PRISM® Optical Cover Compression Pad (Applied Biosystems), transferred to a GeneAmp® PCR System 9700 thermocycler (Applied Biosystems), and the 24 different first amplification products were generated in each of the four parallel first amplification compositions using a thermal profile of 96° C. for 5 minutes to activate the polymerase, ten cycles of 94° C. for thirty seconds, 60° C. for forty-five seconds, and 72° C. for forty-five seconds, then 72° C. for ten minutes, then cooled to 4-10° C.

Step 2: Purifying the Multiplicity of Different First Amplification Products (PCR Clean-Up).

To degrade unincorporated primers and dNTPs, 2 μL ExoSAP-IT® reagent (USB Corporation, Cleveland, Ohio) was added to each of the four first amplification compositions comprising first amplification products. The plate was covered with MicroAmp Adhesive Film and an ABI PRISM® Optical Cover Compression Pad, transferred to a 9700 thermocycler and incubated at 37° C. for thirty minutes, then at 80° C. for fifteen minutes, then cooled to 4° C. The plate was centrifuged at 99 ×g for 1 minute in a Juan CRP 422 centrifuge, then half of the volume was removed from each well and frozen. The remaining purified first amplification compositions were diluted 1:5 with Molecular Biology grade nuclease-free water (“nuclease-free water”; Sigma-Aldrich, St. Louis Mo.).

Step 3: Massively Parallel Single-Plex PCR (Second Amplification Reaction).

Twenty-four different second amplification compositions were formed for each of the four purified first amplification products in wells of a MicroAmp 96-well plate (Applied Biosystems, Foster City, Calif.). Each of the four second amplification compositions comprised 5 μL AmpliTaq Gold® PCR Master Mix (Applied Biosystems P/N 4318739), 3 μL of a first amplification product primer set specific for one of the 24 different first amplification products (0.4 μM forward primer, 0.4 μM reverse primer), 1.6 μL glycerol (50%), and 0.4 μL of one of the diluted, purified different first amplification products from Step 2. The plate was covered with MicroAmp Adhesive Film and an ABI PRISM® Optical Cover Compression Pad (Applied Biosystems), transferred to a GeneAmp® PCR System 9700 thermocycler (Applied Biosystems), and a second amplification product was generated in each of the single-plex second amplification compositions using a thermal profile of 96° C. for 5 minutes to activate the polymerase, 40 cycles of (94° C. for thirty seconds, 60° C. for forty-five seconds, and 72° C. for forty-five seconds), then 72° C. for ten minutes. The four twenty-four single-plex second amplification compositions were then cooled to 4-10° C.

Step 4: Sequencing using Reporter Group-Labeled Terminators (Third Amplification Reaction).

Two sequencing master mixes were prepared by combining 400 μL BigDye® Terminator Ready Reaction Mix v3.1 (Applied Biosystems P/N 4337454), 300 μL nuclease-free water, and 100 μL (3.2 pmol/μL) of either −21 M13 forward primers, 5′TGTAAAACGACGGCCAGT (SEQ ID NO:49) or −21 M13 reverse primers, 5′CAGGAAACAGCTATGACC (SEQ ID NO:50). Two MicroAmp® 96-well plates were used for the sequencing reaction, one for forward sequencing and one for reverse sequencing. Eight μL of the sequencing master mix comprising the M13 forward primer was transferred to wells of the “forward” 96 well plate and 8 μL of the sequencing master mix comprising the M13 reverse primer was transferred to wells of the “reverse” plate. Two μL of each of the second amplification composition comprising the second amplification products were added to appropriate wells of each plate. The plates were covered with Adhesive Films and Optical Cover Compression Pads and centrifuged at 99×g for 1 minute in a Juan centrifuge to ensure that all components were mixed. Third amplification products were generated by cycle sequencing in a GeneAmp® 9700 PCR System thermocycler using a temperature profile of 96° C. for one minute, twenty-five cycles of (96° C. for ten seconds, 50° C. for 5 seconds, and 60° C. for 4 minutes), then the plates were cooled to 4° C.

To each well containing third amplification products, 2.5μL EDTA (125 mM) and 30 μL ethanol (100%) were added and the plates were sealed with Adhesive Film and agitated to mix. The plates were covered and incubated at room temperature for 15 minutes. The liquid was removed from the plates and 30 μL ethanol (70%) was added to each well. The plates were again covered with Adhesive Film or aluminum foil tape and centrifuged at 2830×g for 15 seconds in a Juan centrifuge. The covers were removed from the plates and the ethanol wash discarded by spinning the plates inverted at 99×g for one minute onto a paper towel. The plates were air dried at room temperature for 15 minutes, then sealed with aluminum sealing tape (#6570, Corning Inc. Life Sciences) until the third amplification products in each well were sequenced. For sequencing, 10 μL of HiDi formamide (Applied Biosystems) was added to each well. The plates were covered with Adhesive Film, briefly vortexed and then the resuspended third amplification products were loaded into 36 cm capillaries containing POP-7™ polymer in an Applied Biosystems 3730xI DNA Analyzer using the standard sequencing run module for a 36 cm array (e.g., injection time: 15 seconds; injection voltage: 1.2 kvolt; run time: 1540; run temperature: 60° C.).

The DNA yield after this third amplification reaction was evaluated using a PicoGreen dsDNA Quantification Kit (#11495, Molecular Probes, Eugene, Oreg.), according to the manufacturer's protocol. An aliquot was also electrophoresed on an E-gel from Invitrogen (Carlsbad, Calif.) for evaluation.

### EXAMPLE 2

### gDNA Isothermal Amplification and Sequencing Method Comprising Four Amplification Reactions

The same 24 illustrative target regions were amplified and resequenced using the same 4 gDNA samples as described in Example 1 according to the following exemplary method comprising four amplification reactions. All work was performed on ice when possible, unless otherwise noted.

Step 1: PCR (First Amplification Reaction).

Two sets of 24 different target-specific primer pairs were synthesized. One set of target-specific primer pairs comprised a forward target-specific primer and a reverse target-specific primer, each comprising (a) a target-binding portion comprising target flanking region-specific sequences, i.e., the same sequence as the first target flanking sequence of the gDNA target region or a sequence that is complementary with the second target flanking sequence of the gDNA target region, located at the 3′-end of the primer, and (b) a tail comprising a primer-binding site comprising an M13 universal priming sequence, TGTAAAACGACGGCCAGT (SEQ ID NO:51), located upstream of the target-binding portion of the primer. Each of the forward target-specific primers further comprised a T7 promoter sequence: TAATACGACTCACTATAGGGAGA (SEQ ID NO:52), located upstream from the primer-binding site of the forward target-specific primer (“Primer Set 1”). The second set of target-specific primer pairs comprised a forward target-specific primer, a second forward primer, and a reverse target-specific primer. The forward target-specific primer and reverse target-specific primer of each primer set comprised (a) a first or second target-binding portion, as appropriate and (b) a tail comprising a primer-binding site comprising the M13 sequence, TGTAAAACGACGGCCAGT (SEQ ID NO:51), located upstream of the target-binding portion. The second forward primer of each target-specific primer set was a universal primer comprising a primer-binding site comprising an M13 sequence and a T7 promoter sequence, TAATACGACTCACTATAGGGAGATGTAAAACGACGGCCAGT (SEQ ID NO:53), located upstream from the corresponding primer-binding site (“Primer Set 2”). Thus, incorporation of the sequences of Primer Set 1 into corresponding amplicons is completed in one stage, while incorporation of the sequences of Primer Set 2 into corresponding amplicons is completed in two stages, the first stage incorporating the first forward primer and the corresponding reverse primer and the second stage incorporating the universal second forward primer.

Four first amplification master mixes were formed, each comprising 500 μL AmpliTaq Gold® PCR Master Mix (Applied Biosystems P/N 4318739), 100 μL of one of the four different gDNA samples (1 ng/μL), 160 μL glycerol (50%), and 40 μL nuclease-free water. Eight μL of a first amplification master mix was added to appropriate wells of two ABI PRISM® 96-Well Optical Reaction Plate (Applied Biosystems P/N 4326659). Two μL Primer Set 1 or Primer Set 2 was added to appropriate wells on the plates. The plates were sealed with MicroAmp® Clear Adhesive Films (Applied Biosystems P/N 4306311), mixed briefly by vortexing with a VWR Scientific Products MVI mini-vortex, then centrifuged at 99 ×g for one minute. The plates were covered with MicroAmp® Full Plate Covers (Applied Biosystems P/N N801-0550) and transferred to a GeneAmp® PCR System 9700 thermocycler. First amplification products were generated in the plate comprising Primer Set 1 using a thermal profile of 96° C. for 5 minutes to activate the polymerase, forty cycles of (94° C. for thirty seconds, 60° C. for forty-five seconds, and 72° C. for forty-five seconds), then 72° C. for ten minutes, and finally the plate was cooled to 4-10° C. First amplification products were generated in the plate comprising Primer Set 2 using a thermal profile of 96° C. for 5 minutes to activate the polymerase, ten cycles of (94° C. for thirty seconds, 60° C. for forty-five seconds, and 72° C. for forty-five seconds), thirty cycles of (94° C. for thirty seconds, 55° C. for forty-five seconds, and 72° C. for forty-five seconds), then 72° C. for ten minutes, and finally the plate was cooled to 4-10° C. The quality of the first amplification products was evaluated using an agarose gel.

Step 2: Purifying the First Amplification Products (PCR Clean-Up).

This step was performed as described in Step 2 of Example 1, except that all of the first amplification compositions comprising first amplification products were diluted by adding 18 μL of water to each well.

Step 3: In Vitro Transcription (Second Amplification Reaction).

Second amplification compositions were formed in 0.2 mL thin wall RNase free tubes (Ambion #12225) by combining 2 μL ATP solution (75 mM ATP), 2 μL CTP solution (75 mM CTP), 2 μL GTP solution (75 mM GTP), 2 μL UTP solution (75 mM UTP), 2 μL 10× reaction buffer, 2 μL T7 RNA polymerase mix (all from a MEGAscript® High Yield Transcription Kit, Ambion, Austin Tex.), 7 μL nuclease-free water, and 1 μL diluted, purified first amplification composition, generated in Step 2. The tubes were transferred to a GeneAmp® PCR System 9700 thermocycler and incubated at 37° C. for 6 hours to generate cRNA (second amplification products).

Step 4: Purifying the Second Amplification Products (IVT Clean Up).

The second amplification products were purified using a QIAgen RNeasy Mini Kit (Qiagen, Valencia, Calif.). Ten μL of 2-mercaptoethanol (2-ME) was added to every 1 mL of Buffer RLT before use. The reaction compositions comprising the cRNA from Step 3 were each adjusted to 100 μL using RNase-free water, then 350 μL of the Buffer RLT containing 2-ME was added to each tube with mixing. Next, 250 μL ethanol (100%) was added to each tube and mixed by pipetting, then the contents from a tube (˜700 μL) was applied to a RNeasy spin column in a collection tube. The spin column was centrifuged for 15 seconds at 11,000 rpm in a Microfuge® 18 (Beckman Coulter), then the spin column was transferred to a new 2 mL collection tube. The column was washed with 500 μL Buffer RPE and centrifuging for 15 seconds at 11,000 rpm. The flow-through was discarded and a second 500 μL aliquot of Buffer RPE was added to the column. The column was centrifuged at maximum speed for 2 minutes to dry and the flow-through was discarded. The column was transferred to a new 1.5 mL collection tube. The purified second amplification products were eluted form the column by a first elution with 50 μL RNase-free water and centrifugation at 11,000 rpm for 1 minute, followed by a second elution with 30 μL RNase-free water and centrifugation at 11,000 for an additional minute. The quality of the cRNA was evaluated by electrophoresing 2 μL of the column eluate on a 1 % denaturing gel. The cRNA concentration was determined by measuring the OD 260 and A280 on a Gene Spec III spectrophotometer (Hitachi Genetic Systems).

Step 5: cDNA Synthesis (Third Amplification Reaction).

The purified cRNA from Step 4 was converted to cDNA for sequencing using a SuperScript™ Double-Stranded cDNA Synthesis Kit (Invitrogen P/N 11917-010). A third amplification composition comprising 1 μL M13 universal reverse primer (3.2 μM) comprising the sequence: 5′CAGGAAACAGCTATGACC (SEQ ID NO:53), 2 μL purified cRNA (1 μg/μL in nuclease-free water), 1 μL dNTPs (10 mM), and 8 μL RNase-free water was formed in a RNase-free 1.5 mL tube. The tube was incubated at 65° C. for 5 minutes, then placed on ice for 2 minutes. An RT master mix was prepared by combining 4 μL 5× First Strand Buffer, 2 μL DTT (0.1 M), and 1 μL Rnase out. The RT master mix was added to the composition. The tube was incubated at 42° C. for 2 minutes, then 1 μL SuperScript II polymerase (200 U/μL) was added. The tube was mixed by vortexing briefly, then briefly centrifuged. First strand synthesis was carried out by incubating the tube at 42° C. for 50 minutes and 70° C. for 15 minutes, then the tube was placed on ice.

Step 6: Purifying the cDNA Third Amplification Product (RT Clean-Up).

To degrade the cRNA of the cRNA:cDNA duplex, 1 μL RNase H (2 U/μL; Invitrogen), 1 μL RNase A (100 U/μL; Qiagen), and 1 μL shrimp alkaline phosphatase (SAP, USB Corporation) were added to tubes comprising the cRNA:cDNA amplification products. The cRNA was degraded by incubating the tube at 37° C. for 30 minutes, 80° C. for 15 minutes, 37° C. for 5 minutes, and then cooled to 4° C.

Step 7: Sequencing using Reporter Group-Labeled Nucleotide Terminators (Fourth Amplification Reaction).

Cycle sequencing was performed and the nucleotide sequences were obtained as described in Step 4 of Example 1, except that the cDNA from Step 6 was used as templates in the fourth amplification compositions. Those in the art will appreciate that the single-stranded cDNA can be converted to double-stranded DNA by a PCR or second strand synthesis reaction using appropriate primers and that either or both strands of the resulting double-stranded amplification product can be sequenced using methods known in the art. Alternatively, an RT-PCR reaction can be performed as Step 5, for example, to generate double-stranded amplification products; or an asymmetric RT-PCR reaction can be performed to generate both single-stranded and double-stranded amplification products, either or both of which can be sequenced.

Although the disclosed teachings have been described with reference to various applications, methods, and compositions, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

TABLE 1

| | |
|---|---|
| Target Region ID | Target-Specific Primer Sets |

| | | |
|---|---|---|
| hCG14702_103 | F: TGTAAAACGACGGCCAGTCTGGAGCCATGAGCGTGTCC | (SEQ ID NO:1) |
| | R: CAGGAAACAGCTATGACCGGCCACAAATGGGAGCACAG | (SEQ ID NO:2) |
| hCG14702_106 | F: TGTAAAACGACGGCCAGTCCATCAGCTTCCAGAGGCCC | (SEQ ID NO:3) |
| | R: CAGGAAACAGCTATGACCAGAAGTCCAGCCCACCTGCG | (SEQ ID NO:4) |
| hCG14715_35 | F: TGTAAAACGACGGCCAGTCCACCCAGGTGTAACTTGCCA | (SEQ ID NO:5) |
| | R: CAGGAAACAGCTATGACCGGAAGTTAACAGGGTGTAGACAAGGGA | (SEQ ID NO:6) |
| hCG16028_166 | F: TGTAAAACGACGGCCAGTCGAACCAGCTGGGAATGCAC | (SEQ ID NO:7) |
| | R: CAGGAAACAGCTATGACCTGCAACTGAAAGAGGGTTGCCA | (SEQ ID NO:8) |
| hCG16028_174 | F: TGTAAAACGACGGCCAGTGCCTCGCAGTCAGTTTCTCCC | (SEQ ID NO:9) |
| | R: CAGGAAACAGCTATGACCTCAGAAACCCAAGCCACTCCA | (SEQ ID NO:10) |
| hCG16377_164 | F: TGTAAAACGACGGCCAGTTGCTTCTGCTTCCATGTGCTTTC | (SEQ ID NO:11) |
| | R: CAGGAAACAGCTATGACCAGGCTTTCCCACAGTACTTGCAT | (SEQ ID NO:12) |
| hCG27692_65 | F: TGTAAAACGACGGCCAGTTCCGAGGTGCTTGGGAGTTT | (SEQ ID NO:13) |
| | R: CAGGAAACAGCTATGACCCATGCCACTTTGGCTTGTATATTGTC | (SEQ ID NO:14) |
| hCG1810776_143 | F: TGTAAAACGACGGCCAGTGCCGAGTGACATGGGCACAG | (SEQ ID NO:15) |
| | R: CAGGAAACAGCTATGACCTGCAGCTCCTTCTTTATGTCAGCA | (SEQ ID NO:16) |
| hCG1814900_50 | F: TGTAAAACGACGGCCAGTTGCCTTTGATGTGGCTGTTGG | (SEQ ID NO:17) |
| | R: CAGGAAACAGCTATGACCCGGGACCTGCCCTCTCCA | (SEQ ID NO:18) |
| hCG1816688_61 | F: TGTAAAACGACGGCCAGTTCTGCCTGTCCACCTATTCTCACA | (SEQ ID NO:19) |
| | R: CAGGAAACAGCTATGACCGCACAACCTGTCAGATGCCAGAC | (SEQ ID NO:20) |
| hCG1816688_62 | F: TGTAAAACGACGGCCAGTGCATTTATCTCGTGCCGAATGG | (SEQ ID NO:21) |
| | R: CAGGAAACAGCTATGACCTCCAGCATTGGACTAACGTGGTT | (SEQ ID NO:22) |
| hCG1816688_64 | F: TGTAAAACGACGGCCAGTTCCTTCCTGGTAGGCCTGATTCAT | (SEQ ID NO:23) |
| | R: CAGGAAACAGCTATGACCATGCACCCACGCTCCTTACG | (SEQ ID NO:24) |
| hCG1817253_23 | F: TGTAAAACGACGGCCAGTTCAGCATACACTCATTCCTTTCCGA | (SEQ ID NO:25) |
| | R: CAGGAAACAGCTATGACCGGCAGTCTCTGGCTGTGGGA | (SEQ ID NO:26) |
| hCG1817253_24 | F: TGTAAAACGACGGCCAGTATCCAGCCTCCCAGGCTCAG | (SEQ ID NO:27) |
| | R: CAGGAAACAGCTATGACCTGGTGTTGAGGAAACTGAGTGGC | (SEQ ID NO:28) |
| hCG1817253_39 | F: TGTAAAACGACGGCCAGTCCCTGAGGGATTACAAGCAAGGG | (SEQ ID NO:29) |
| | R: CAGGAAACAGCTATGACCCCAGCCGCAAACATGGAAAC | (SEQ ID NO:30) |
| hCG1818580_161 | F: TGTAAAACGACGGCCAGTTGGAAATGAGAAGAACTCAAGTGTGG | (SEQ ID NO:31) |
| | R: CAGGAAACAGCTATGACCTTCGGAGGTGAATGGGAGCC | (SEQ ID NO:32) |
| hCG1818580_162 | F: TGTAAAACGACGGCCAGTCACCATCTTTCACTCACCTCGAT | (SEQ ID NO:33) |
| | R: CAGGAAACAGCTATGACCTGAAATTGGTAATGTCAACTGTTCTGG | (SEQ ID NO:34) |
| hCG37044_20 | F: TGTAAAACGACGGCCAGTCACCTTGCCACTCATTCCTTGA | (SEQ ID NO:35) |
| | R: CAGGAAACAGCTATGACCACGGGAGGTGCAAGTGACCA | (SEQ ID NO:36) |
| hCG37044_22 | F: TGTAAAACGACGGCCAGTTGGTCACTTGCACCTCCCGT | (SEQ ID NO:37) |
| | R: CAGGAAACAGCTATGACCGGGAAATAACTTGTGCAAAGGCG | (SEQ ID NO:38) |
| hCG37044_24 | F: TGTAAAACGACGGCCAGTTCCAGGATATTCCTACCCAGGGC | (SEQ ID NO:39) |
| | R: CAGGAAACAGCTATGACCGCCCAGCTGTGGTCATTGGA | (SEQ ID NO:40) |
| hCG37044_25 | F: TGTAAAACGACGGCCAGTAGGGTCCCGGGAAACTTGC | (SEQ ID NO:41) |
| | R: CAGGAAACAGCTATGACCGACACATCAAGGTTGCCCTTCC | (SEQ ID NO:42) |
| hCG37044_27 | F: TGTAAAACGACGGCCAGTGGAAGGGCAACCTTGATGTGTC | (SEQ ID NO:43) |
| | R: CAGGAAACAGCTATGACCGGTTTCTCCATGTTGGTCAGGC | (SEQ ID NO:44) |
| hCG37044_28 | F: TGTAAAACGACGGCCAGTTCAGCGCTCACCTTGAAGCC | (SEQ ID NO:45) |
| | R: CAGGAAACAGCTATGACCGGCGGAAGTGCAATGGTGAA | (SEQ ID NO:46) |
| hCG37044_42 | F: TGTAAAACGACGGCCAGTCACCATTCCAGCCTGGGAGTC | (SEQ ID NO:47) |
| | R: CAGGAAACAGCTATGACCAAGGTGAGCGCTGAGCCAGA | (SEQ ID NO:48) |

(F = forward primer; R = reverse primer)

We claim:

1. A method for determining the sequence of a genomic DNA (gDNA) target region comprising:
forming a first amplification composition comprising the gDNA, a target-specific primer set, a first extending enzyme, and nucleoside triphosphates (NTPs), wherein the target specific primer set comprises (a) a forward target-specific primer comprising (i) a first target-binding portion that comprises a sequence that is the same as a first target flanking region and (ii) an upstream tail portion comprising a first primer-binding site, a first promoter sequence, or a first primer-binding site and a first promoter sequence and (b) a corresponding reverse target-specific primer comprising (i) a second target-binding portion that comprises a sequence that is complementary to a corresponding second target flanking region and (ii) an upstream tail portion comprising a second primer-binding site, a second promoter sequence, or a second primer-binding site and a second promoter sequence;
amplifying the gDNA in the first amplification composition to generate a first amplification product;
forming a second amplification composition comprising at least some of the first amplification product, a second extending enzyme, and NTPs;
amplifying the first amplification product in the second amplification composition to generate a second amplification product; and
sequencing at least part of the second amplification product to determine the sequence of the target region.

2. The method of claim 1, wherein the sequencing comprises sequencing by hybridization, chemical cleavage, restriction mapping, mass spectrometry, capillary electrophoresis, or combinations thereof.

3. The method of claim 1, wherein the sequencing comprises determining (a) the identity of a polymorphic nucleotide at a SNP site in the target region and/or (b) the presence or absence of a mutation in the target region.

4. The method of claim 1, wherein the first extending enzyme and the second extending enzyme are the same or different.

5. The method of claim 1, wherein at least one of the target-specific primers, at least one the first amplification products, at least one of the second amplification products, or combinations thereof, further comprises a hybridization tag, an affinity tag, a reporter group, or combinations thereof.

6. The method of claim 1, wherein the sequencing comprises forming a third amplification composition comprising at least some of the second amplification product, a DNA-dependent DNA polymerase, a second amplification product primer, and NTPs, wherein the NTPs comprise a deoxyribonucleotide triphosphate (dNTP), a nucleotide terminator, or a dNTP and a nucleotide terminator; amplifying the second amplification product in the third amplification composition to generate a third amplification product; and obtaining the nucleotide sequence of at least part of the second amplification product, at least part of the third amplification product, or at least part of the second amplification and at least part of the third amplification product to determine the sequence of the target region.

7. The method of claim 6, wherein the second amplification product primer comprises a second amplification product primer set comprising a forward second amplification product primer and a reverse second amplification product primer.

8. The method of claim 6, further comprising purifying: (a) the first amplification product before the amplifying the first amplification product, (b) the second amplification product before the amplifying the second amplification product, (c) the third amplification product before obtaining the sequence of the at least part of the third amplification product, or (d) combinations thereof; wherein the purifying comprises: (a) degrading an unincorporated primer, an unincorporated NTP, or an unincorporated primer and an unincorporated NTP, and/or (b) separating the amplification product from an unincorporated primer, an unincorporated NTP, or an unincorporated primer and an unincorporated NTP.

9. The method of claim 8, wherein the first extending enzyme comprises a DNA-dependent DNA polymerase, the NTPs of the first amplification composition comprise dNTPs, and the target-specific primer comprises a multiplicity of different target-specific primer sets; the second extending enzyme comprises a DNA-dependent DNA polymerase, the NTPs of the second amplification composition comprise dNTPs, and wherein the second amplification composition further comprises a first amplification product primer set comprising a forward first amplification product primer and a reverse first amplification product primer; and the third extending enzyme comprises a DNA-dependent DNA polymerase and the NTPs of the third amplification composition comprise a dNTP, a nucleotide terminator, or a dNTP and a nucleotide terminator.

10. The method of claim 9, wherein the amplifying the gDNA comprises a multiplex polymerase chain reaction (PCR) and the amplifying the first amplification product comprises a single-plex PCR.

11. The method of claim 10, wherein the single-plex PCR comprises a multiplicity of different single-plex PCR reactions, each in a different second amplification composition comprising at least some of the first amplification product, a second extending enzyme, and a first amplification product primer.

12. The method of claim 6, wherein the obtaining the nucleotide sequence comprises sequencing by hybridization, chemical cleavage, restriction mapping, mass spectrometry, capillary electrophoresis, or combinations thereof.

13. The method of claim 6, wherein the fourth amplification composition further comprises an ATP sulfurylase and a luciferase.

14. The method of claim 6, wherein at least one of the target-specific primers, at least one of the first amplification product primers, at least one the first amplification products, at least one of the second amplification products, at least one of the third amplification products, or combinations thereof, further comprises a hybridization tag, an affinity tag, a reporter group, or combinations thereof.

15. A method for determining the sequence of a gDNA target region comprising:
forming a first amplification composition comprising the gDNA, a target-specific primer set, a first extending enzyme, and dNTPs, wherein the target-specific primer set comprises (a) a forward target-specific primer comprising (i) a first target-binding portion that comprises a sequence that is the same as a first target flanking region and (ii) an upstream tail portion comprising a first primer-binding site, a first promoter sequence, or a first primer-binding site and a first promoter sequence and (b) a corresponding reverse target-specific primer comprising (i) a second target-binding portion that comprises a sequence that is complementary to a corresponding second target flanking region and (ii) an upstream tail portion comprising a second primer-binding site, a second promoter sequence, or a second primer-binding site and a second promoter sequence;
amplifying the gDNA in the first amplification composition to generate a first amplification product,
forming a second amplification composition comprising at least some of the first amplification product, a second extending enzyme, and NTPs;
amplifying the first amplification product in the second amplification composition to generate a second amplification product;
forming a third amplification composition comprising at least some of the second amplification, a third extending enzyme, and NTPs;
amplifying the second amplification product in the third amplification composition to generate a third amplification product;
contacting the third amplification product with a third amplification product primer;
amplifying the third amplification product to generate a fourth amplification product; and
sequencing at least part of the fourth amplification product to determine the sequence of the target region.

16. The method of claim 15, wherein the sequencing comprises sequencing by hybridization, chemical cleavage, restriction mapping, pyrosequencing, mass spectrometry, capillary electrophoresis, or combinations thereof.

17. The method of claim 15, wherein the sequencing comprises determining (a) the identity of a polymorphic nucleotide at a SNP site in the target region and/or (b) the presence or absence of a mutation in the target region.

18. The method of claim 15, wherein at least one of the third amplification product primers and/or at least one of the fourth amplification products further comprises a hybridization tag, an affinity tag, a reporter group, or combinations thereof.

19. The method of claim 15, wherein the sequencing comprises forming a fourth amplification composition comprising at least some of the fourth amplification product, a DNA-dependent DNA polymerase, a fourth amplification product primer, and a dNTP, a nucleotide terminator, or a dNTP and a nucleotide terminator; amplifying the fourth amplification product in the fourth amplification composition to generate a fifth amplification product; and obtaining the nucleotide sequence of at least part of the fourth amplification product, at least part of the fifth amplification product, or at least part of the fourth amplification and at least part of the fifth amplification product to determine the sequence of the target region.

20. The method of claim 19, wherein the fourth amplification product primer comprises a fourth amplification product primer set comprising a forward fourth amplification product primer and a reverse fourth amplification product primer.

21. The method of claim 19, further comprising purifying: (a) the first amplification product before the amplifying the first amplification product, (b) the second amplification product before the amplifying the second amplification product, (c) the third amplification product before the amplifying the fourth amplification product, (d) the fifth amplification product before the obtaining the sequence of the at least a part of the fifth amplification product, or (e) combinations thereof; wherein the purifying comprises: (a) degrading an unincorporated primer, an unincorporated NTP, or an unincorporated primer and an unincorporated NTP, and/or (b) separating the amplification product from an unincorporated primer, an unincorporated NTP, or an unincorporated primer and an unincorporated NTP.

22. The method of claim 19, wherein the first extending enzyme comprises a DNA-dependent DNA polymerase, the NTPs of the first amplification composition comprise dNTPs; the second extending enzyme comprises a DNA-dependent RNA polymerase, and the NTPs of the second amplification composition comprise rNTPs; the third extending enzyme comprises an RNA-dependent DNA polymerase, a DNA-dependent DNA polymerase or an RNA-dependent DNA polymerase and a DNA-dependent DNA polymerase, and the NTPs of the third amplification composition comprise dNTPs; and the fourth extending enzyme comprises a DNA-dependent DNA polymerase and the NTPs of the fourth amplification composition comprise a dNTP, a nucleotide terminator, or a dNTP and a nucleotide terminator.

23. The method of claim 19, wherein the obtaining the nucleotide sequence comprises sequencing by hybridization, chemical cleavage, restriction mapping, mass spectrometry, capillary electrophoresis, or combinations thereof.

24. The method of claim 19, wherein the fourth amplification composition further comprises an ATP sulfurylase and a luciferase.

25. The method of claim 19, wherein at least one of the third amplification product primers, at least one of the fourth amplification product primers, at least one the fourth amplification products, at least one of the second amplification products, at least one of the fourth amplification products, at least one of the fifth amplification products, or combinations thereof, further comprises a hybridization tag, an affinity tag, a reporter group, or combinations thereof.

26. A method for determining the sequence of a multiplicity of different gDNA target regions comprising:
forming a first amplification composition comprising the gDNA, a multiplicity of different target-specific primer sets, a first DNA-dependent DNA polymerase, and dNTPs, wherein each target specific primer set comprises (a) a first forward target-specific primer comprising (i) a first target-binding portion that comprises a sequence that is the same as a first target flanking region and (ii) a first tail portion comprising a first primer-binding site, and (b) a corresponding reverse target-specific primer comprising (i) a second target-binding portion that comprises a sequence that is complementary to a second target flanking region and (ii) a second tail portion comprising a second primer binding site;
amplifying the gDNA in the first amplification composition using a PCR comprising 5-15 amplification cycles to generate a multiplicity of different first amplification products;
purifying the multiplicity of different first amplification products;
forming a second amplification composition comprising at least some of the purified first amplification product, a second DNA-dependent DNA polymerase, a first amplification product primer set, and dNTPs, wherein the first amplification primer set comprises (a) a forward primer comprising a sequence that is the same as the first primer-binding site of the corresponding first forward primer and (b) a reverse primer comprising a sequence that is complementary with the second primer-binding site of the corresponding first reverse primer;
amplifying the first amplification product in the second amplification composition to generate a second amplification product; and
sequencing at least part of the second amplification product, wherein the sequencing comprises (a) forming a third amplification composition comprising at least some of the second amplification product, a third DNA-dependent DNA polymerase, a second amplification product primer, a reporter group-labeled ddNTP or a dNTP and a reporter group-labeled ddNTP; (b) amplifying the second amplification product in the third amplification composition to generate a reporter group-labeled third amplification product; (c) purifying the reporter group-labeled third amplification product; and (d) obtaining the nucleotide sequence of at least part of the purified third amplification product using capillary electrophoresis to determine the sequence of at least two of the different gDNA target regions.

27. The method of claim 26, wherein the third product primer comprises a third product primer set comprising a forward third amplification product primer and a reverse third product amplification product primer.

28. A method for determining the sequence of a gDNA target region comprising:
forming a first amplification composition comprising the gDNA, a target-specific primer set, a first extending enzyme, and dNTPs, wherein the target-specific primer set comprises (a) a forward target-specific primer comprising (i) a first target-binding portion comprising a sequence that is the same as a first target flanking region and (ii) a first tail portion comprising a first primer-binding site and (b) a corresponding target-specific reverse primer comprising (i) a second target-binding portion comprising a sequence that is complementary with a corresponding second target flanking region and (ii) a second tail portion comprising a second primer binding site;
amplifying the gDNA in the first amplification composition to generate a first amplification product;
forming a second amplification composition comprising at least some of the first amplification product, a DNA-dependent RNA polymerase, and rNTPs;
amplifying the first amplification product in the second amplification composition to generate a second amplification product;
forming a third amplification composition comprising at least some of the second amplification, an RNA-dependent DNA polymerase, a DNA-dependent DNA polymerase, or an RNA-dependent DNA polymerase and a DNA-dependent DNA polymerase, and dNTPs;
amplifying the second amplification product in the third amplification composition to generate a third amplification product;
contacting the third amplification product with a third amplification product primer;
amplifying the third amplification product to generate a fourth amplification product; and
sequencing at least part of the fourth amplification product, wherein the sequencing comprises forming a fourth amplification composition comprising at least some of the fourth amplification product, a DNA-dependent DNA polymerase, a fourth amplification product primer, and a reporter group-labeled nucleotide terminator or a dNTP and a reporter group-labeled nucleotide terminator; amplifying the fourth amplification product in the fourth amplification composition to generate a reporter-group-labeled fifth amplification product; and obtaining the nucleotide sequence of at least part of the fifth amplification product using capillary electrophoresis comprising laser-induced fluorescence to determine the sequence of the gDNA target region.

29. The method of claim 28, wherein the fourth product primer comprises a fourth amplification product primer set comprising a forward fourth amplification product primer and a reverse fourth product amplification product primer.

30. The method of claim 28, wherein the first tail portion of the forward target-specific primer further comprises a first promoter sequence.

31. The method of claim 28, wherein the target-specific primer set further comprises a second forward primer comprising (i) a sequence that is complementary with the first primer-binding sequence of the forward target-specific primer and (ii) a third tail portion comprising a promoter sequence.

32. A kit for determining the sequence of at least one gDNA target region comprising a first DNA-dependent DNA polymerase, a second DNA-dependent DNA polymerase, a DNA-dependent RNA polymerase, an RNA-dependent DNA polymerase, a target-specific primer set for each gDNA target region, a nucleotide terminator, and a sequencing primer.

33. The kit of claim 32, wherein the second DNA-dependent DNA polymerase and the RNA-dependent DNA polymerase comprise the same extending enzyme.

34. The kit of claim 32, further comprising a third DNA-dependent DNA polymerase.


SEQUENCE LISTING (53 sequences)

SEQ ID NO: 1 - DNA, 38 bp, Artificial Sequence
  Description of Artificial Sequence Synthetic primer
tgtaaaacgacggccagtctggagccatgagcgtgtcc

SEQ ID NO: 2 - DNA, 38 bp, Artificial Sequence
  Description of Artificial Sequence Synthetic primer
caggaaacagctatgaccggccacaaatgggagcacag

SEQ ID NO: 3 - DNA, 38 bp, Artificial Sequence
  Description of Artificial Sequence Synthetic primer
tgtaaaacgacggccagtccatcagcttccagaggccc

SEQ ID NO: 4 - DNA, 38 bp, Artificial Sequence
  Description of Artificial Sequence Synthetic primer
caggaaacagctatgaccagaagtccagcccacctgcg

SEQ ID NO: 5 - DNA, 39 bp, Artificial Sequence
  Description of Artificial Sequence Synthetic primer
tgtaaaacgacggccagtccacccaggtgtaacttgcca

SEQ ID NO: 6 - DNA, 45 bp, Artificial Sequence
  Description of Artificial Sequence Synthetic primer
caggaaacagctatgaccggaagttaacagggtgtagacaaggga

SEQ ID NO: 7 - DNA, 38 bp, Artificial Sequence
  Description of Artificial Sequence Synthetic primer
tgtaaaacgacggccagtcgaaccagctgggaatgcac

SEQ ID NO: 8 - DNA, 40 bp, Artificial Sequence
  Description of Artificial Sequence Synthetic primer
caggaaacagctatgacctgcaactgaaagagggttgcca

SEQ ID NO: 9 - DNA, 39 bp, Artificial Sequence
  Description of Artificial Sequence Synthetic primer
tgtaaaacgacggccagtgcctcgcagtcagtttctccc

SEQ ID NO: 10 - DNA, 39 bp, Artificial Sequence
  Description of Artificial Sequence Synthetic primer
caggaaacagctatgacctcagaaacccaagccactcca

SEQ ID NO: 11 - DNA, 41 bp, Artificial Sequence
  Description of Artificial Sequence Synthetic primer
tgtaaaacgacggccagttgcttctgcttccatgtgctttc

SEQ ID NO: 12 - DNA, 41 bp, Artificial Sequence
  Description of Artificial Sequence Synthetic primer
caggaaacagctatgaccaggctttcccacagtacttgcat

SEQ ID NO: 13 - DNA, 38 bp, Artificial Sequence
  Description of Artificial Sequence Synthetic primer
tgtaaaacgacggccagttccgaggtgcttgggagttt

SEQ ID NO: 14 - DNA, 44 bp, Artificial Sequence
  Description of Artificial Sequence Synthetic primer
caggaaacagctatgacccatgccactttggcttgtatattgtc

SEQ ID NO: 15 - DNA, 38 bp, Artificial Sequence
  Description of Artificial Sequence Synthetic primer
tgtaaaacgacggccagtgccgagtgacatgggcacag

SEQ ID NO: 16 - DNA, 42 bp, Artificial Sequence
  Description of Artificial Sequence Synthetic primer
caggaaacagctatgacctgcagctccttctttatgtcagca

SEQ ID NO: 17 - DNA, 39 bp, Artificial Sequence
  Description of Artificial Sequence Synthetic primer
tgtaaaacgacggccagttgcctttgatgtggctgttgg

SEQ ID NO: 18 - DNA, 36 bp, Artificial Sequence
  Description of Artificial Sequence Synthetic primer
caggaaacagctatgacccgggacctgccctctcca

SEQ ID NO: 19 - DNA, 42 bp, Artificial Sequence
  Description of Artificial Sequence Synthetic primer
tgtaaaacgacggccagttctgcctgtccacctattctcaca

SEQ ID NO: 20 - DNA, 41 bp, Artificial Sequence
  Description of Artificial Sequence Synthetic primer
caggaaacagctatgaccgcacaacctgtcagatgccagac

SEQ ID NO: 21 - DNA, 40 bp, Artificial Sequence
  Description of Artificial Sequence Synthetic primer
tgtaaaacgacggccagtgcatttatctcgtgccgaatgg

SEQ ID NO: 22 - DNA, 41 bp, Artificial Sequence
  Description of Artificial Sequence Synthetic primer
caggaaacagctatgacctccagcattggactaacgtggtt

SEQ ID NO: 23 - DNA, 42 bp, Artificial Sequence
  Description of Artificial Sequence Synthetic primer
tgtaaaacgacggccagttccttcctggtaggcctgattcat

SEQ ID NO: 24 - DNA, 38 bp, Artificial Sequence
  Description of Artificial Sequence Synthetic primer
caggaaacagctatgaccatgcacccacgctccttacg

SEQ ID NO: 25 - DNA, 43 bp, Artificial Sequence
  Description of Artificial Sequence Synthetic primer
tgtaaaacgacggccagttcagcatacactcattcctttccga

SEQ ID NO: 26 - DNA, 38 bp, Artificial Sequence
  Description of Artificial Sequence Synthetic primer
caggaaacagctatgaccggcagtctctggctgtggga

SEQ ID NO: 27 - DNA, 38 bp, Artificial Sequence
  Description of Artificial Sequence Synthetic primer
tgtaaaacgacggccagtatccagcctcccaggctcag

SEQ ID NO: 28 - DNA, 41 bp, Artificial Sequence
  Description of Artificial Sequence Synthetic primer
caggaaacagctatgacctggtgttgaggaaactgagtggc

SEQ ID NO: 29 - DNA, 41 bp, Artificial Sequence
  Description of Artificial Sequence Synthetic primer
tgtaaaacgacggccagtccctgagggattacaagcaaggg

SEQ ID NO: 30 - DNA, 38 bp, Artificial Sequence
  Description of Artificial Sequence Synthetic primer
caggaaacagctatgaccccagccgcaaacatggaaac

SEQ ID NO: 31 - DNA, 44 bp, Artificial Sequence
  Description of Artificial Sequence Synthetic primer
tgtaaaacgacggccagttggaaatgagaagaactcaagtgtgg

SEQ ID NO: 32 - DNA, 38 bp, Artificial Sequence
  Description of Artificial Sequence Synthetic primer
caggaaacagctatgaccttcggaggtgaatgggagcc

SEQ ID NO: 33 - DNA, 41 bp, Artificial Sequence
  Description of Artificial Sequence Synthetic primer
tgtaaaacgacggccagtcaccatctttcactcacctcgat

SEQ ID NO: 34 - DNA, 45 bp, Artificial Sequence
  Description of Artificial Sequence Synthetic primer
caggaaacagctatgacctgaaattggtaatgtcaactgttctgg

SEQ ID NO: 35 - DNA, 40 bp, Artificial Sequence
  Description of Artificial Sequence Synthetic primer
tgtaaaacgacggccagtcaccttgccactcattccttga

SEQ ID NO: 36 - DNA, 38 bp, Artificial Sequence
  Description of Artificial Sequence Synthetic primer
caggaaacagctatgaccacgggaggtgcaagtgacca

SEQ ID NO: 37 - DNA, 38 bp, Artificial Sequence
  Description of Artificial Sequence Synthetic primer
tgtaaaacgacggccagttggtcacttgcacctcccgt

SEQ ID NO: 38 - DNA, 41 bp, Artificial Sequence
  Description of Artificial Sequence Synthetic primer
caggaaacagctatgaccgggaaataacttgtgcaaaggcg

SEQ ID NO: 39 - DNA, 41 bp, Artificial Sequence
  Description of Artificial Sequence Synthetic primer
tgtaaaacgacggccagttccaggatattcctacccagggc

SEQ ID NO: 40 - DNA, 38 bp, Artificial Sequence
  Description of Artificial Sequence Synthetic primer
caggaaacagctatgaccgcccagctgtggtcattgga

SEQ ID NO: 41 - DNA, 37 bp, Artificial Sequence
  Description of Artificial Sequence Synthetic primer
tgtaaaacgacggccagtagggtcccgggaaacttgc

SEQ ID NO: 42 - DNA, 40 bp, Artificial Sequence
  Description of Artificial Sequence Synthetic primer
caggaaacagctatgaccgacacatcaaggttgcccttcc

SEQ ID NO: 43 - DNA, 40 bp, Artificial Sequence
  Description of Artificial Sequence Synthetic primer
tgtaaaacgacggccagtggaagggcaaccttgatgtgtc

SEQ ID NO: 44 - DNA, 40 bp, Artificial Sequence
  Description of Artificial Sequence Synthetic primer
caggaaacagctatgaccggtttctccatgttggtcaggc

SEQ ID NO: 45 - DNA, 38 bp, Artificial Sequence
  Description of Artificial Sequence Synthetic primer
tgtaaaacgacggccagttcagcgctcaccttgaagcc

SEQ ID NO: 46 - DNA, 38 bp, Artificial Sequence
  Description of Artificial Sequence Synthetic primer
caggaaacagctatgaccggcggaagtgcaatggtgaa

SEQ ID NO: 47 - DNA, 39 bp, Artificial Sequence
  Description of Artificial Sequence Synthetic primer
tgtaaaacgacggccagtcaccattccagcctgggagtc

SEQ ID NO: 48 - DNA, 38 bp, Artificial Sequence
  Description of Artificial Sequence Synthetic primer
caggaaacagctatgaccaaggtgagcgctgagccaga

SEQ ID NO: 49 - DNA, 18 bp, Artificial Sequence
  Description of Artificial Sequence Synthetic primer
tgtaaaacgacggccagt

SEQ ID NO: 50 - DNA, 18 bp, Artificial Sequence
  Description of Artificial Sequence Synthetic primer
caggaaacagctatgacc

SEQ ID NO: 51 - DNA, 18 bp, Artificial Sequence
  Description of Artificial Sequence Synthetic oligonucleotide
tgtaaaacgacggccagt

SEQ ID NO: 52 - DNA, 23 bp, Artificial Sequence
  Description of Artificial Sequence Synthetic oligonucleotide
taatacgactcactatagggaga

SEQ ID NO: 53 - DNA, 41 bp, Artificial Sequence
  Description of Artificial Sequence Synthetic oligonucleotide
taatacgactcactatagggagatgtaaaacgacggccagt